(19) United States
(12) Patent Application Publication
(10) Pub. No.: US 2023/0091105 A1
(43) Pub. Date: Mar. 23, 2023

(54) CHIMERIC FUSIONS BETWEEN C4-BINDING PROTEIN C-TERMINAL SEGMENT AND ANGIOPOIETIN-1 FIBRINOGEN-LIKE DOMAIN AS ANGIOPOIETIN MIMETICS AND TIE2 AGONISTS TO TREAT VASCULAR DISEASES

(71) Applicants: Northwestern University, Chicago, IL (US); Mannin Research Inc., Toronto (CA)

(72) Inventors: Jing JIN, Chicago, IL (US); Pan LIU, Chicago, IL (US); Michael RYCZKO, Toronto (CA)

(21) Appl. No.: 17/802,670

(22) PCT Filed: Jun. 25, 2020
(86) PCT No.: PCT/US2020/039554

§ 371 Date: Aug. 26, 2022

Related U.S. Application Data
(60) Provisional application No. 62/983,328, filed on Feb. 28, 2020.
(60) Provisional application No. 63/029,369, filed on May 22, 2020.

(51) Int. Cl. C07K 14/515; C07K 14/47; C07K 14/55; C07K 14/705; A61K 39/395; A61K 38/17; A61K 31/7088; A61K 31/517; A61K 31/502; A61K 31/404; A61K 38/18; A61P 9/14

(52) U.S. Cl. CPC C07K 14/515; C07K 14/4703; C07K 14/55; C07K 14/70503; A61K 39/3955; A61K 38/179; A61K 31/7088; A61K 38/1709; A61K 31/517; A61K 31/502; A61K 31/404; A61K 38/1891; A61P 9/14; C07K 2319/70; C07K 2319/21; C07K 2319/02; C07K 2319/23; C07K 2319/24; C07K 2319/20; C07K 2319/50

(57) ABSTRACT

The disclosure relates to Angiopoietin-1 mimetics for treating vascular diseases via agonistic activation of Tie2/TEK receptor.

### SEQUENCE LISTING

The instant application contains a Sequence Listing which has been submitted electronically in ASCII format and is hereby incorporated by reference in its entirety. Said ASCII copy, created on Jun. 17, 2020, is named 14002_6006-00134_SL.txt and is 57,523 bytes in size.

### FIELD

The disclosure relates, among other aspects, to Angiopoietin-1 mimetics for treating vascular diseases via agonistic activation of Tie2/TEK receptor.

### BACKGROUND

The Angiopoietin-Tie2 signaling pathway is a major regulator of vascular development, vessel remodeling, post-natal angiogenesis, and vessel permeability (Saharinen P, Eklund L, Alitalo K. Therapeutic targeting of the angiopoietin-TIE pathway. Nat Rev Drug Discov. 2017; 16(9):635-661). This pathway mainly operates through direct binding of endothelial tyrosine kinase receptor Tie2 (TEK) by its extracellular ligands Angiopoietin-1 (Ang1) and 2 (Ang2) (Saharinen P, Eklund L, Alitalo K. Therapeutic targeting of the angiopoietin-TIE pathway. Nat Rev Drug Discov. 2017; 16(9):635-661). While the strong canonical agonist function of Ang1 is well defined, Ang2 is often considered a context-dependent antagonist of Tie2 (Souma T, et al. Context-dependent functions of angiopoietin 2 are determined by the endothelial phosphatase VEPTP. Proc Natl Acad Sci USA. 2018; 115(6):1298-1303). In addition, the strength of Ang-Tie2 signaling is modulated by negative regulators such as vascular endothelial protein tyrosine phosphatase (VEPTP/PTPRB), and the pathway also has crosstalk with Integrin signaling (Saharinen P, Eklund L, Alitalo K. Therapeutic targeting of the angiopoietin-TIE pathway. Nat Rev Drug Discov. 2017; 16(9):635-661). Downstream of Tie2, numerous intracellular signal transduction pathways can be activated, leading to ERK1/2, AKT and eNOS phosphorylation (Saharinen P, Eklund L, Alitalo K. Therapeutic targeting of the angiopoietin-TIE pathway. Nat Rev Drug Discov. 2017; 16(9):635-661).

The Angiopoietin-Tie2 signaling system has been studied as a potential therapeutic target for treating a broad range of diseases. There is a large body of literature describing how activating this pathway has protective effects against vascular leakage and inflammation (Parikh S M. Angiopoietins and Tie2 in vascular inflammation. Curr Opin Hematol. 2017; 24(5):432-438; Saharinen P, Eklund L, Alitalo K. Therapeutic targeting of the angiopoietin-TIE pathway. Nat Rev Drug Discov. 2017; 16(9):635-661). Indications include but not limited to cancer, sepsis, ischemic stroke, acute kidney injury, chronic kidney disease, diabetic nephropathy and retinopathy, wound healing, acute lung injury, allograft rejection, among other diseases and conditions (Saharinen P, Eklund L, Alitalo K. Therapeutic targeting of the angiopoietin-TIE pathway. Nat Rev Drug Discov. 2017; 16(9):635-661). Modulating this pathway through exogenous intervention provides a therapeutic opportunity to stabilize vascular endothelium by preventing detrimental effects of inflammation and vascular leakage, thereby preserving endothelial barrier integrity (Parikh S M. Angiopoietins and Tie2 in vascular inflammation. Curr Opin Hematol. 2017; 24(5):432-438).

There has been considerable effort by academic laboratories and biotechnology companies to generate bioequivalent or biobetter Ang analogues or mimetics for therapeutic use. Several designs of Ang1 mimetics have been attempted, however none has reached clinical trials stage primarily due to obstacles encountered in achieving desired pharmacological effects (Koh G Y. Orchestral actions of angiopoietin-1 in vascular regeneration. Trends Mol Med. 2013; 19(1):31-39).

Angiopoietins share similar molecular domain architecture, having a C-terminal fibrinogen-like domain (FLD)—which confers binding to the cell surface receptor Tie2, a middle coiled-coil domain (CCOD)—which mediates homo-multimerization of monomers, and a shorter N-terminal super-clustering domain (SCD) segment—which enables clustering of Angiopoietin dimers into multimeric structures through intramolecular disulfide bridges (FIG. 1A) (Koh G Y. Orchestral actions of angiopoietin-1 in vascular regeneration. Trends Mol Med. 2013; 19(1):31-39). Higher oligomerization is a major determinant of potency and while monomeric Angiopoietin ligands can bind Tie2, they do not induce Tie2 receptor tyrosine phosphorylation and activation of downstream intracellular signaling that regulate microvasculature and is crucial for blood and lymphatic vessel development, maintenance and function (Saharinen P, Eklund L, Alitalo K. Therapeutic targeting of the angiopoietin-TIE pathway. Nat Rev Drug Discov. 2017; 16(9):635-661). Ang1 is a potent agonist of Tie2 that predominantly exists in higher-order multimeric forms, which promotes clustering of Tie2 receptors and elicits downstream signaling cascades (Koh G Y. Orchestral actions of angiopoietin-1 in vascular regeneration. Trends Mol Med. 2013; 19(1):31-39). Higher-order multimeric ligands are optimal binders of Tie2 and due to avidity strongly induce tyrosine phosphorylation of ligand-complexed Tie2 receptors (Kim K T, et al. Oligomerization and multimerization are critical for angiopoietin-1 to bind and phosphorylate Tie2. J Biol Chem. 2005; 280(20):20126-20131). In contrast, Ang2 most frequently exists as a dimer, making it a competitive antagonist of Tie2 when in the presence of Ang1, but a partial agonist of Tie2 in the relative absence of Ang1 and VE-PTP, which appears to set up the threshold for Tie2 responsiveness to each ligand (Souma T, et al. Context-dependent functions of angiopoietin 2 are determined by the endothelial phosphatase VEPTP. Proc Natl Acad Sci USA. 2018; 115(6):1298-1303). In addition to differences in multimerization and Tie2 engagement, Ang1 binds to extracellular matrix and hyaluronan, the main structural component of the endothelial glycocalyx (van den Berg B M, et al. Glomerular Function and Structural Integrity Depend on Hyaluronan Synthesis by Glomerular Endothelium. J Am Soc Nephrol. 2019; 30(10):1886-1897).

Native Ang1 is mainly produced by vascular pericytes. It binds the extracellular matrix (ECM) via its N-terminus domain and linker, and through the C-terminus Tie2-binding fibrinogen-like domain (FLD) activates Tie2 receptor on the adjacent endothelium (Koh G Y. Orchestral actions of angiopoietin-1 in vascular regeneration. Trends Mol Med. 2013; 19(1):31-39). This mode of action makes it challenging to achieve systemic drug efficacy using a native form of Ang1. Recombinant Ang1 available as experimental reagent from biotechnology vendors is produced as heterogeneous multimers of trimeric, tetrameric and pentameric oligomers (Koh G Y. Orchestral actions of angiopoietin-1 in vascular regeneration. Trends Mol Med. 2013; 19(1):31-39). Due to its unique molecular structure, SCD-CCOD has an intrinsic tendency to be sticky, bind non-specifically to ECM, and form insoluble aggregates, resulting in precipitation and loss of activity (Koh G Y. Orchestral actions of angiopoietin-1 in vascular regeneration. Trends Mol Med. 2013; 19(1):31-39). Therefore, native Ang1 form is not considered a good drug candidate. To circumvent these problems several Ang1-mimetics have been bioengineered using different designs to attempt to improve solubility, stability and multimericity. One approach used a design that replaced SCD-CCOD with a dimerizing fragment crystallizable (Fc) from IgG1 to create Bow-ANG1, which had a low multimericity of 2 (Davis S, et al. Angiopoietins have distinct modular domains essential for receptor binding, dimerization and superclustering. Nat Struct Biol. 2003; 10(1):38-44). To increase multimericity, an alternative version of BOW-ANG1 was constructed with two FLDs placed in each chain in a tandem arrangement to boost multimericity to 4, which displayed an enhanced binding affinity to Tie2 receptor (Davis S, et al. Angiopoietins have distinct modular domains essential for receptor binding, dimerization and superclustering. Nat Struct Biol. 2003; 10(1):38-44). Another approach used a shorter and more stable CCOD from cartilage oligomeric matrix protein fused to the FLD, generating a pentamer referred to as COMP:Ang1 that can strongly activate Tie2 (Cho C H, et al. Designed angiopoietin-1 variant, COMP-Ang1, protects against radiation-induced endothelial cell apoptosis. Proc Natl Acad Sci USA. 2004; 101(15):5553-5558.). Even though Bow-Ang1 and COMP:Ang1 do show some efficacy in activating Tie2 in vivo, their shortcomings such as non-specific binding to extracellular matrix and short blood half-life in the case of COMP-Ang1, and low-multimericity and weak potency of BOW-Ang1 render them unsuitable for clinical trials (Koh G Y. Orchestral actions of angiopoietin-1 in vascular regeneration. Trends Mol Med. 2013; 19(1):31-39). Therefore, there remains a need to create Ang1 mimetics with improved solubility, stability and multimericity.

The Complement binding protein (C4BP) is an abundant human plasma glycoprotein whose natural function is to inhibit the classical and lectin pathways of complement activation (Ermert D, Blom A M. C4b-binding protein: The good, the bad and the deadly. Novel functions of an old friend. Immunol Lett. 2016; 169:82-92). With the predominant form in human blood composed of seven identical alpha chains and a single beta chain, C4BP assumes a seven-arm spider or octopus-like structure held together at the C-terminal end (Hofmeyer T, et al. Arranged sevenfold: structural insights into the C-terminal oligomerization domain of human C4b-binding protein. J Mol Biol. 2013; 425(8):1302-1317). This C-terminal core region is responsible for assembly into a multimer during protein synthesis, with cysteine from one monomer forming intermolecular disulfide bond with the cystine of another monomer (Hofmeyer T, et al. Arranged sevenfold: structural insights into the C-terminal oligomerization domain of human C4b-binding protein. J Mol Biol. 2013; 425(8):1302-1317). C4BP scaffold is sufficient to oligomerize full-length C4BP, has a remarkable stability, and tolerates well harsh conditions such as exposure to extreme pH and temperature (Hofmeyer T, et al. Arranged sevenfold: structural insights into the C-terminal oligomerization domain of human C4b-binding protein. J Mol Biol. 2013; 425(8):1302-1317). In a chimeric fusion, C4BP is also predicted to be able to oligomerize other linked domains, and here we describe C4BP fusions with Ang1 (FIG. 1B).

### SUMMARY

Through rational design, herein described is a new “biobetter” mimetic of Angiopoietin-1 (ANG1) that can be used as an injectable therapeutic for treatment of vascular conditions through Tie2 activation. The disclosure relates to the design, construction, production and therapeutic use of chimeric fusions between ANG1's C-terminus Tie2-binding fibrinogen-like domain (FLD) and the C-terminus scaffold segment of Complement C4-Binding Protein (C4BP). The recombinant fusion, referred to as either ANG1-C4BP or C4BP-ANG1 based on their N-to-C terminus order of domain arrangement, naturally folds into a heptameric structure via the C4BP segment and displays 7 FLDs of ANG1 in a “bouquet of tulips”-like configuration (FIG. 1B), resembling that of native ANG1 (FIG. 1A). Recombinant produced ANG1-C4BP and C4BP-ANG1 potently activate Tie2 in human cells and mouse models. Aspects of the disclosure also relate to cell lines expressing such recombinant fusion proteins and to methods of decreasing or inhibiting vascular leakage or plasma permeability, and promoting growth and maintaining structural integrity of vasculature. Exemplary intended indications of therapeutic use of ANG1-C4BP series of biologics include vascular eye diseases, such as primary open angle glaucoma caused by defects in limbus capillary plexus or Schlemm's canal drainage system, and types of primary or secondary retinopathy, as well as for systemic treatment of vascular leakage as in cancer neoangiogenesis, conditions of inflammation, among others.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference.

Other features and advantages of the disclosure will be apparent from the Drawings and the following Detailed Description, including the Examples, and the claims.

### BRIEF DESCRIPTIONS OF DRAWINGS

The patent or application file contains at least one drawing executed in color. Copies of this patent or patent application publication with color drawings will be provided by the U.S. Patent and Trademark Office upon request and payment of the necessary fee.

FIG. 1 shows the schematic drawing and the actual formation of heptameric C4BP-ANG1. A) Native ANG1 is comprised of, from an N- to C-terminus order, a supercluster domain (SCD), a coiled-coil domain (CCOD), and a fibrinogen-like domain that binds Tie2 (top). The CCOD mediates CCOD-CCOD interactions between ANG1 molecules (middle), and through its linker segment with FLD also binds the ECM. The SCD further clusters ANG1 into higher degree complexes (bottom). B) The C-terminus of C4BP naturally folds into a “barrel” structure through inter-linking disulfide bridges (red) between neighboring subunits. A total of seven (or eight) of these subunits complete the barrel structure (top) that, in C4BP-ANG1 or ANG1-C4BP, displays seven FLD in an arrangement reminiscent of that of native ANG1 (bottom, compared to A). C) C4BP-ANG1 was expressed through transfection of the encoding plasmid into HEK-293 cells and collected from the culture medium. As expected, C4BP-ANG1 formed a heptamer under non-reducing (NR) condition on SDS PAGE. D. Electron micrograph (EM) images showed clustered C4BP-ANG1.

FIG. 2A and FIG. 2B is a summary of different versions of Angiopoietin and C4BP chimeric fusion constructs generated and expressed in HEK293 and CHO cells.

FIG. 3 shows expression of ANG1-C4BP heptamers by CHO and HEK293 in culture media. Transient expression of various Angiopoietin-C4 bp fusion constructs in both CHO and HEK293 (three transfection conditions 1-3 tested) cells. Constructs H6EKC4BPAng1 and H6EKAng1C4BP expressed at highest levels with the correct formation of ˜280 kDa heptamers (upper panel), and ˜35 kDa monomer (lower panel) under reducing condition as shown with A) Ponceau S solution staining under non-reduced and reduced conditions, and B) non-reducing and reducing SDS PAGE western blots using anti-His-Tag antibody.

FIG. 4 shows C4BP and ANG1 fusion variants all form heptamers in near homogeneity (part 1). In an N-to-C-terminus order, 4 plasmids for mammalian cell expression were constructed: 1. C4BP-ANG1(1) with a C-terminus 6×His tag (SEQ ID NO: 30), 2. C4BP-ANG(2) with an N-terminus 6×His tag (SEQ ID NO: 30), 3. ANG1-C4BP(1) with a C-terminus 6×His tag (SEQ ID NO: 30), and 4. ANG1-C4BP(2) with an N-terminus 6×His tag (SEQ ID NO: 30). Proteins were expressed in CHO cells cultured in serum-free medium and subsequently harvested from the culture medium. By performing SDS PAGE analysis under either non-reducing (N.R.: left panel) or reducing (right panel) conditions, it was determined that C4BP-ANG1 proteins (highlighted in red boxes), regardless of their N-to-C orders, naturally form heptamers (with multiplicity of 7) of ˜280 kDa via disulfide bridges. All fusion proteins can be reduced to their ˜35 kDa monomeric forms under reducing condition.

FIG. 5 shows purified ANG1-C4BP variants forming heptamers (part 2). Products of chimeric fusion proteins were found at the expected molecular weight in all constructs following non-reduced SDS PAGE separation and western blot analysis using anti-His-Tag antibody. #2 denotes use of an alternative expression vector for C4BPAng1H6 for comparison. CHO-BRI stable pool expression platform technology was used to produce these ANG-C4BP variants.

FIG. 6 shows purified ANG1-C4BP variants forming heptamers (part 3). The products of chimeric fusion proteins were found at the expected molecular weight in all constructs following non-reduced SDS-PAGE separation and western blot analysis using anti-His-Tag antibody. #2 denotes use of an alternative expression vector for C4BPANG1H6 for comparison. CHO-BRI stable pool expression platform technology was used to produce these ANG-C4BP variants.

FIG. 7 shows IMAC purification of peak #2 containing heptamers of ANG1-C4BP variants. A) Non-reduced and reduced SDS PAGE Coomassie blue stain of IMAC purified fractions. Peak #2 has the correct molecular weight for the recombinant fusion protein products—heptamer formation under non-reduced and monomer formation under reduced conditions. B) An overview of IMAC purified factions, highlighting peak #2.

FIG. 8 shows ANG1-C4BP chimeric fusion protein stability following freeze-thaw cycles. Purified ANG1-C4BP was subjected to one or two freeze-thaw cycles (F/T) before UPLC-SEC analysis of heptamer quality (at peak 2.610). No loss of the heptamer fraction was apparent (compare 1 F/T and 2 F/T with the control that was stored at 4° C.).

FIG. 9 shows binding of ANG1-C4BP and C4BP-ANG1 to Tie2. Using the ectodomain of Tie2 in the form of an Fc fusion (Tie2-Fc), direct interactions between Tie2 and recombinant ANG1 (rANG1) of native ANG1 sequence, ANG1-C4BP or C4BP-ANG1 were tested in a co-immunoprecipitation assay. Following anti-Fc immunoprecipitation, the presence of the ANG1 variants was detected by anti-His tag blotting. The immunoblotting images were from a composite double staining with anti-His and anti-Fc antibodies.

FIG. 10 shows ANG1-C4BP activates Tie2 in a dose-dependent manner in cultured HUVEC. Increase in phosphorylation level of AKT (pAKT) observed in HUVEC following treatment for 20 minutes with pre-prep-SEC peak #2 of ANG1-C4BP. The half-maximal response (EC₅₀) for Ang1-C4 bp in activating pAKT in HUVEC treated for 20 minutes was 87 ng/mL.

FIG. 11 shows ANG1-C4BP variants activate Tie2 in a dose-dependent manner. Chimeric fusion between ANG1 and C4BP are potent agonists of Tie2 receptor in vitro, as evidence by A) increase in phosphorylation of Tie2 and B) its downstream target AKT. The experiment was performed in HUVEC with concentrations indicated or 500 ng/mL of each recombinant chimeric fusion protein as treatment for 20 minutes. rhAngpt1 is recombinant Angiopoietin-1 from R&D Systems.

FIG. 12 shows C4BP-ANG1 induces relocalization of Tie2 to loci in cell periphery. HUVEC cells were transgene transfected with FLAG-Tie2 (full length) and subjected to vehicle control or C4BP-ANG1 treatment. Tie2 images in green were developed from anti-FLAG immunofluorescence staining (a representative single cell image from each group is shown).

FIG. 13 shows i.v. and i.p. injection of C4BP-ANG1 activates endogenous Tie2 in mice. Mice were injected with C4BP-ANG1 and in vivo activities were measured by phosphorylation of endogenous Tie2 (pTyr-Tie2) in the lung. A) Mice were i.v. injected with either vehicle or C4BP-ANG1 of different doses based on body weight (BW) and lung tissues were harvested 30 minutes after. Following anti-Tie2 immunoprecipitation, phospho-Tie2 levels were measured by immunoblotting with anti-pTyr antibody. B) and C) show time course studies of phospho-Tie2 in response to C4BP-ANG1 at 0.5 μg/g.BW.

FIG. 14 shows pharmacokinetics of intravitreous injected C4BP-ANG1 in rabbit eye. Three rabbits were each subjected to a single dose of intravitreal injection of C4BP-ANG1 and aqueous humor was collected daily (preinjection sample: day 0) for seven days. The levels of C4BP-ANG1 were measured by ELISA using anti-His capturing antibody and anti-ANG1 detection antibody (OD450 values) (left). On the seventh day the animals were sacrificed and vitreous humor samples were collected for detection of C4BP-ANG1 levels (right, asterisks: p<0.01).

FIG. 15 shows C4BP-ANG1 reduces VEGF-induced vascular leakage in Miles assay in mice. The studies of vascular leakage were conducted using Miles assay, which quantifies tissue level of Evans Blue dye. Mice were subjected to a 30 min injection schedule as shown (top). Subcutaneous (SQ) injections of a combination of VEGF and C4BP-ANG1 were performed and leakage of Evans Blue was visualized (bottom) and quantified as OD360 values normalized by tissue weight (image and quantification, right asterisks: p<0.001).

FIG. 16 shows i.v. injection of C4BP-ANG1 reduces VEGF-induced vascular leakage. The studies of vascular leakage were conducted using the Miles assay, which quantifies tissue levels of Evans Blue dye. Mice were subjected to a 30 min injection schedule as shown (top). Instead of local injection of C4BP-ANG1, the biologic was administrated prophylactically via i.v. 60 minutes prior to leakage induction by subcutaneous (SQ) injections of VEGF. Leakage of Evans Blue was visualized (bottom).

FIG. 17 shows i.v. injection of C4BP-ANG1 reduces chemical-induced vascular leakage. The studies of vascular leakage were conducted using Miles assay, which quantifies tissue levels of Evans Blue dye. Injection of C4BP-ANG1 was administrated prophylactically via i.v. 60 minutes prior to leakage induction by topical application of mustard oil to the ear (image and quantification, asterisks: p<0.01).

FIG. 18 shows C4BP-ANG1 protects from lipopolysaccharide-induced lung injury in mice. In a mouse model of lipopolysaccharide(LPS)-induced lung injury, a time course of LPS inhalation (INH), C4BP-ANG1 injection, and Evans Blue injection was followed as indicated in the top panel. One hour after Evans Blue injection, the lungs were harvested to measure vascular leakage (image and quantification, asterisk: p<0.05).

FIG. 19 shows expression, purification and in vitro and in vivo Tie2 activation of tag-less Ang1C4 bp construct. The expression construct contains a signal peptide, followed by Ang1 FLD, a “GGGS” linker (SEQ ID NO: 31) and the C4 bp sequence in an N-to-C-terminus order. Using CHO cell system, the tag-less Ang1-linker-C4 bp fusion is expressed and secreted into the culture medium. A) Following ion-exchange chromatography, protein peaks were eluted off the column (left). Non-reducing SDS PAGE analysis of the collections showed target tag-less protein Ang1C4 bp was concentrated in fractions F4 and F5 (right panel: highlighted in red boxes and size indicated by a red arrow). B) Fractions F4 and F5 were combined and loaded onto a size-exclusion chromatography column for “polishing” of the target protein, which resulted in further enrichment (left panel: chromatogram tracing shows target protein, indicated by the red arrow; right panel: SDS PAGE confirmed the successful enrichment of the target protein in fraction F2, indicated by the red arrow). C) Treatment of HUVEC and HEK293 cells (stably expressing Tie2-FLAG transgene) with tag-less Ang1C4 bp in activated intracellular Akt phosphorylation (pAkt) and Tie2 phosphorylation (pTie2), respectively. Vehicle control (Ctr) and native Ang1 were used as negative and positive controls, respectively. D) Mice were i.v. injected with tag-less Ang1C4 bp recombinant protein to induced Tie2 phosphorylation in the lung. Lung tissues from vehicle injection (Ctr: n=3) and from tag-less Ang1C4 bp injection (n=3) were harvested 1 hour after injection. Total Tie2 was immunoprecipitated (IP) from the lung tissue homogenates (anti-Tie2 IP) and Tie2 phosphorylation levels were determined by immunoblotting of the IP samples with anti-phosphotyrosine antibody (pTie2).

### DETAILED DESCRIPTION

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and.”

The term “and/or” where used herein is to be taken as specific disclosure of each of the two specified features or components with or without the other. Thus, the term “and/or” as used in a phrase such as “A and/or B” herein is intended to include A and B; A or B; A (alone), and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114,115, 116, 117, 118, 119, 120, 121, 122, 123, 124, 125, 126, 127,128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” may mean within one or more than one standard deviation per the practice in the art. “About” or “comprising essentially of” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Binding affinity” generally refers to the strength of the sum total of non-covalent interactions between a single binding site of a molecule (e.g., of an antibody) and its binding partner (e.g., an antigen). Unless indicated otherwise, as used herein, “binding affinity”, “bind to”, “binds to” or “binding to” refers to intrinsic binding affinity that reflects a 1:1 interaction between members of a binding pair (e.g., antibody Fab fragment and antigen). The affinity of a molecule X for its partner Y can generally be represented by the dissociation constant (KD). Affinity may be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which may be used for purposes of the present invention. The Label-free surface plasmon resonance (SPR)-based biosensors, such as BIACORE methods, and MM/PBSA methods, and KinExA are standard methods often preferred. It is known that the binding affinities can change depending on the assay. Accordingly, for purposes of this disclosure, it is sufficient that the binding affinity fall within the recited range when measured by at least one method standard in the art.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed., (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Chimeric polypeptides, nucleic acids and host cells of the present description, and (pharmaceutical) compositions thereof, may be administered to a subject in need thereof by routes known in the art, and may vary depending on the use, for example, the type of ocular disease to be treated. In one embodiment, the administration is intravenous injection, intraperitoneal injection, subcutaneous injection, intravitreal injection. In one embodiment, routes of administration include, for example, local administration (such as intraocular) and parenteral administration such as subcutaneous, intraperitoneal, intramuscular, intravenous, intraportal and intrahepatic. In a preferred embodiment, Chimeric polypeptides, nucleic acids or host cells of the present disclosure, or pharmaceutical compositions thereof, are administered to a subject by local infusion, for example using an infusion pump and/or catheter system, to a site to be treated, such as a solid tumor. In one embodiment, a composition of the present description is infused into a solid tumor, a blood vessel that feeds a solid tumor, and/or the area surrounding a solid tumor. Other exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intra-arterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal, or mucosal route of administration, for example, intranasally, vaginally, rectally, sublingually or topically. Administering may also be performed, for example, once, a plurality of times, and/or over one or more extended periods.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrases “determining an amount” of an analyte and the like may be used. Where a qualitative and/or quantitative determination is intended, the phrase “determining a level” of an analyte or “detecting” an analyte is used.

The terms “recombinant host cell” or “host cell” refer to a cell into which exogenous, e.g., recombinant, DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. coli; mammalian cell lines CHO, HEK 293, COS, NS0, SP2 and PER.C6; the insect cell line Sf9; and the fungal cell Saccharomyces cerevisiae.

“Vector” refers to a polynucleotide capable of being duplicated within a biological system or that may be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems. “Expression vector” refers to a vector that may be utilized in a biological system or in a reconstituted biological system to direct the translation of a polypeptide encoded by a polynucleotide sequence present in the expression vector. “Expression vector” refers to a vector that may be utilized in a biological system or in a reconstituted biological system to direct the translation of a polypeptide encoded by a polynucleotide sequence present in the expression vector.

Any range disclosed herein is intended to encompass the endpoints of that range unless stated otherwise. Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reference” on “control” is meant a standard of comparison. The standard may be an established method in the art. A control reference method is a reference method in which all of the parameters are identical to those of the method being compared with exception of the variable being tested. It may also be the average value for the parameter being measured from what is typically used or known in the art.

Numerous types of competitive binding assays may be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland et al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15), solid phase direct biotin-avidin EIA (Cheung, et al., 1990, Virology 176:546-552), and direct labeled RIA (Moldenhauer et al., 1990, Scand. J. Immunol. 32:77-82).

A “therapeutically effective amount,” “effective dose,” “effective amount,” or “therapeutically effective dosage” of a therapeutic agent, e.g., engineered chimeric polypeptides, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Dosages of the molecules of the present disclosure may vary between wide limits, depending upon the disease or disorder to be treated, the age and condition of the individual to be treated.

Actual dosage levels of the active ingredients in the pharmaceutical compositions of the present disclosure may be varied so as to obtain an amount of the active ingredient that is effective to achieve the desired therapeutic response for a particular patient, mode of administration, and composition, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved. In general, a suitable daily dose of a compositions of the disclosure will be that amount of the compound that is the lowest dose effective to produce a therapeutic effect. Such an effective dose will generally depend upon the factors described above. The compositions can be administered with medical devices known in the art. Non-limiting embodiments include a needle, a needleless hypodermic injection device, a variable flow implantable infusion apparatus for continuous drug delivery, an osmotic drug delivery system having multi-chamber compartments.

If desired, the effective daily dose of therapeutic compositions may be administered as two, three, four, five, six or more sub-doses administered separately at appropriate intervals throughout the day, optionally, in unit dosage forms. While it is possible for a compound of the present disclosure to be administered alone, it is preferable to administer the compound as a pharmaceutical formulation (composition)

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement. cDNA is a typical example of a synthetic polynucleotide.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity, or recurrence of a symptom, complication or condition, or biochemical indicia associated with a disease. In one embodiment, the terms “treat,” “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In some embodiments, the wherein the one or more symptoms ameliorated are selected from the group consisting of: weakness, fatigue, shortness of breath, easy bruising and bleeding, frequent infections, enlarged lymph nodes, distended or painful abdomen, bone or joint pain, fractures, unplanned weight loss, poor appetite, night sweats, persistent mild fever, and decreased urination. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both.

The term “subject” as used herein includes human and non-human animals. Non-human animals include all vertebrates, e.g., mammals and non-mammals, such as non-human primates, sheep, dogs, cats, cows, horses, chickens, amphibians, and reptiles.

To calculate percent identity, the sequences being compared are typically aligned in a way that gives the largest match between the sequences. One example of a computer program that may be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm.) In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Chimeric Polypeptides/Fusion Proteins

ANG1-C4BP and C4BP-ANG1 refer to chimeric fusions between ANG1 C-terminus FLD and C4BP C-terminus segment in an N-to-C-terminus order, respectively, in either direction. In general, ANG1-C4BP variants refer to both domain arrangement types of ANG1-C4BP and C4BP-ANG1, and that also include all forms of the fusion with different arrangements of linker and tag locations.

In one embodiment, the disclosure relates to the design, construction, production and therapeutic use of chimeric fusions between Angiopoietin-1's C-terminus Tie2-binding fibrinogen-like domain (FLD) and the C-terminus scaffold segment of C4BP. The disclosure provides a new mimetic of Angiopoietin-1 (ANG1) that can be used for treatment of vascular conditions through Tie2 activation. In one embodiment, the disclosure provides a strategy that has hitherto not been explored, by replacing the SCD-CCOD of ANG1 with a segment of C4BP plasma protein in order to gain the capability of free circulation in the circulatory system. In some embodiments, the chimeric fusion protein is a “biobetter” ANG1.

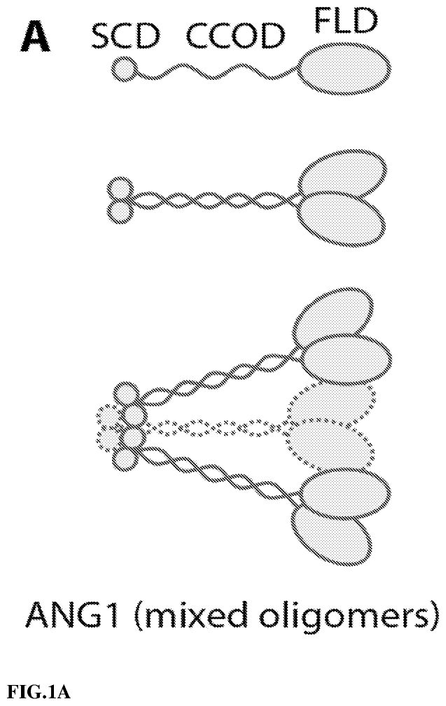

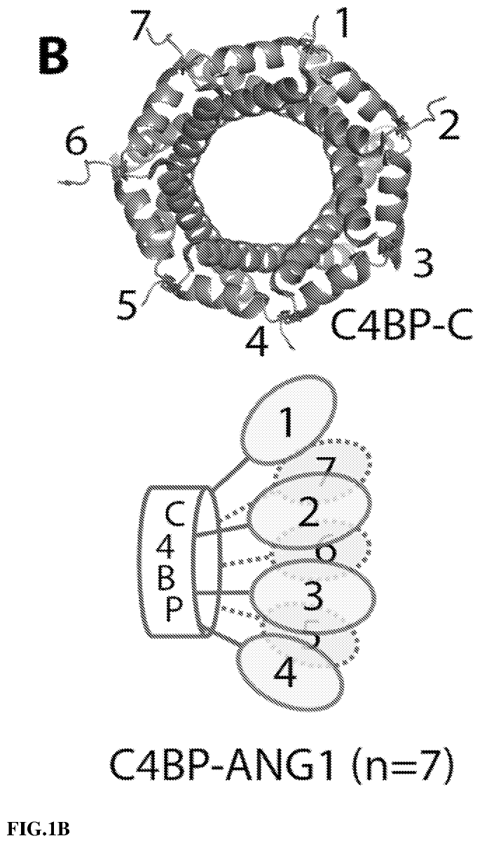

In one embodiment, the disclosure provides that the recombinant fusion, referred to as either ANG1-C4BP or C4BP-ANG1 based on their N-to-C terminus order of domain arrangement, naturally folds into a heptameric structure via the C4BP segment and displays 7 FLDs of ANG1 in a “bouquet of tulips”-like configuration (FIG. 1B), resembling that of native ANG1 (FIG. 1A).

In one embodiment, the C-terminus scaffold segment of human serum C4BP alpha chain was fused with a linker to human ANG1 FLD as C4BP-ANG1 or ANG1-C4BP. In one embodiment, in a chimeric fusion protein with ANG1, the C4BP segment forms a closed ring structure that anchors multimeric C4BP assembly and folds into a stable heptameric central stalk structure that displays seven ANG1 head groups (heptavalent) (FIG. 1). With the design feature of heptameric multimerization through inter-chain disulfide bonding, the seven ANG1 FLDs in the chimeric fusion protein form a high avidity ligand of the cognate Tie2 receptor, resulting in potent binding and agonistic activation of Tie2.

In one embodiment, the recombinant fusion between ANG1 and C4BP may include additional purification tag sequences such as 6×His tag (SEQ ID NO: 30), and with or without an endopeptidase cleavage sequence for tag removal.

In some embodiments, recombinant ANG1-C4BP fusions includes variants with alternative domain arrangements between the ANG1 and the C4BP segments, and the arrangements among these segments, together with additional purification tag and endopeptidase cleavage sequences.

In one embodiment, the C4BP protein comprises the sequence provided in NCBI Accession No. NP_000706.1. In one embodiment, the Angiopoietin 1 protein comprises the sequence provided in NCBI Accession No. NP_001137.2.

In one embodiment, the disclosure provides a polypeptide selected from any one of the following polypeptides and functional fragments or derivatives thereof.

SEQ ID NO.: 0001: c4bp component
ETPEGCEQVLTGKRLMQCLPNPEDVKMALEVYKLSLEIEQLELQRDSARQSTLDKEL
SEQ ID NO.: 0002: Ang1 component
KPFRDCADVYQAGFNKSGIYTIYINNMPEPKKVFCNMDVNGGGWTVIQHREDGSLDFQRGWKEYKMGFGNPSGEYWLGN
EFIFAITSQRQYMLRIELMDWEGNRAYSQYDRFHIGNEKQNYRLYLKGHTGTAGKQSSLILHGADFSTKDADNDNCMCK
CALMLTGGWWFDACGPSNLNGMFYTAGQNHGKLNGIKWHYFKGPSYSLRSTTMMIRPLDF
SEQ ID NO.: 0003: Ang2 component
ISFRDCAEVFKSGHTTNGIYTLTFPNSTEEIKAYCDMEAGGGGWTIIQRREDGSVDFQRTWKEYKVGFGNPSGEYWLGN
EFVSQLTNQQRYVLKIHLKDWEGNEAYSLYEHFYLSSEELNYRIHLKGLTGTAGKISSISQPGNDFSTKDGDNDKCICK
CSQMLTGGWWFDACGPSNLNGMYYPQRQNTNKFNGIKWYYWKGSGYSLKATTMMIRPADF
SEQ ID NO.: 0004: GGGGSlinker
GGGGS
SEQ ID NO.: 0005: IL2 signal peptide
MYRMQLLSCIALSLALVTNS
SEQ ID NO.: 0006: CD33 signal peptide
MPLLLLLPLLWAGALA
SEQ ID NO.: 0007: Enterokinase cleavage site (cleaves after Lysine (K)
DDDDK
SEQ ID NO.: 0008: Ang1-c4bp-H6 (polyHis tag)
KPFRDCADVYQAGFNKSGIYTIYINNMPEPKKVFCNMDVNGGGWTVIQHREDGSLDFQRGWKEYKMGFGNPSGEYWLGN
EFIFAITSQRQYMLRIELMDWEGNRAYSQYDRFHIGNEKQNYRLYLKGHTGTAGKQSSLILHGADFSTKDADNDNCMCK
CALMLTGGWWFDACGPSNLNGMFYTAGQNHGKLNGIKWHYFKGPSYSLRSTTMMIRPLDFGGGGSETPEGCEYKLSLEI
QVLTGKRLMQCLPNPEDVKMALEVEQLELQRDSARQSTLDKELHHHHHH
SEQ ID NO.: 0009: IL2SP-Ang1-c4bp-H6
MYRMQLLSCIALSLALVTNSKPFRDCADVYQAGFNKSGIYTIYINNMPEPKKVFCNMDVNGGGWTVIQHREDGSLDFQR
GWKEYKMGFGNPSGEYWLGNEFIFAITSQRQYMLRIELMDWEGNRAYSQYDRFHIGNEKQNYRLYLKGHTGTAGKQSSL
ILHGADFSTKDADNDNCMCKCALMLTGGWWFDACGPSNLNGMFYTAGQNHGKLNGIKWHYFKGPSYSLRSTTMMIRPLD
FGGGGSETPEGCEQVLTGKRLMQCLPNPEDVKMALEVYKLSLEIEQLELQRDSARQSTLDKELHHHHHH
SEQ ID NO.: 0010: c4bp-Ang1-H6
ETPEGCEQVLTGKRLMQCLPNPEDVKMALEVYKLSLEIEQLELQRDSARQSTLDKELGGGGSKPFRDCADVYQAGFNKS
GIYTIYINNMPEPKKVFCNMDVNGGGWTVIQHREDGSLDFQRGWKEYKMGFGNPSGEYWLGNEFIFAITSQRQYMLRIE
LMDWEGNRAYSQYDRFHIGNEKQNYRLYLKGHTGTAGKQSSLILHGADFSTKDADNDNCMCKCALMLTGGWWFDACGPS
NLNGMFYTAGQNHGKLNGIKWHYFKGPSYSLRSTTMMIRPLDFHHHHHH
SEQ ID NO.: 0011: IL2SP-c4bp-Ang1-H6
MYRMQLLSCIALSLALVTNSETPEGCEQVLTGKRLMQCLPNPEDVKMALEVYKLSLEIEQLELQRDSARQSTLDKELGG
GGSKPFRDCADVYQAGFNKSGIYTIYINNMPEPKKVFCNMDVNGGGWTVIQHREDGSLDFQRGWKEYKMGFGNPSGEYW
LGNEFIFAITSQRQYMLRIELMDWEGNRAYSQYDRFHIGNEKQNYRLYLKGHTGTAGKQSSLILHGADFSTKDADNDNC
MCKCALMLTGGWWFDACGPSNLNGMFYTAGQNHGKLNGIKWHYFKGPSYSLRSTTMMIRPLDFHHHHHH
SEQ ID NO.: 0012: c4bp-Ang2-H6
ETPEGCEQVLTGKRLMQCLPNPEDVKMALEVYKLSLEIEQLELQRDSARQSTLDKELGGGGSISFRDCAEVFKSGHTTN
GIYTLTFPNSTEEIKAYCDMEAGGGGWTIIQRREDGSVDFQRTWKEYKVGFGNPSGEYWLGNEFVSQLTNQQRYVLKIH
LKDWEGNEAYSLYEHFYLSSEELNYRIHLKGLTGTAGKISSISQPGNDFSTKDGDNDKCICKCSQMLTGGWWFDACGPS
NLNGMYYPQRQNTNKFNGIKWYYWKGSGYSLKATTMMIRPADFHHHHHH
SEQ ID NO.: 0013: IL2SP-c4bp-Ang2-H6
MYRMQLLSCIALSLALVTNSETPEGCEQVLTGKRLMQCLPNPEDVKMALEVYKLSLEIEQLELQRDSARQSTLDKELGG
GGSISFRDCAEVFKSGHTTNGIYTLTFPNSTEEIKAYCDMEAGGGGWTIIQRREDGSVDFQRTWKEYKVGFGNPSGEYW
LGNEFVSQLTNQQRYVLKIHLKDWEGNEAYSLYEHFYLSSEELNYRIHLKGLTGTAGKISSISQPGNDFSTKDGDNDKC
ICKCSQMLTGGWWFDACGPSNLNGMYYPQRQNTNKFNGIKWYYWKGSGYSLKATTMMIRPADFHHHHHH
SEQ ID NO.: 0014: H6-EK-Ang1-c4bp
HHHHHHGDDDDKKPFRDCADVYQAGFNKSGIYTIYINNMPEPKKVFCNMDVNGGGWTVIQHREDGSLDFQRGWKEYKMG
FGNPSGEYWLGNEFIFAITSQRQYMLRIELMDWEGNRAYSQYDRFHIGNEKQNYRLYLKGHTGTAGKQSSLILHGADFS
TKDADNDNCMCKCALMLTGGWWFDACGPSNLNGMFYTAGQNHGKLNGIKWHYFKGPSYSLRSTTMMIRPLDFGGGGSET
PEGCEQVLTGKRLMQCLPNPEDVKMALEVYKLSLEIEQLELQRDSARQSTLDKEL
SEQ ID NO.: 0015: IL2SP-H6-EK-Ang1-c4bp
MYRMQLLSCIALSLALVTNSEHHHHHHGDDDDKKPFRDCADVYQAGFNKSGIYTIYINNMPEPKKVFCNMDVNGGGWTV
IQHREDGSLDFQRGWKEYKMGFGNPSGEYWLGNEFIFAITSQRQYMLRIELMDWEGNRAYSQYDRFHIGNEKQNYRLYL
KGHTGTAGKQSSLILHGADFSTKDADNDNCMCKCALMLTGGWWFDACGPSNLNGMFYTAGQNHGKLNGIKWHYFKGPSY
SLRSTTMMIRPLDFGGGGSETPEGCEQVLTGKRLMQCLPNPEDVKMALEVYKLSLEIEQLELQRDSARQSTLDKEL
SEQ ID NO.: 0016: H6-EK-c4bp-Ang1
HHHHHHGGDDDDKETPEGCEQVLTGKRLMQCLPNPEDVKMALEVYKLSLEIEQLELQRDSARQSTLDKELGGGGSKPFR
DCADVYQAGFNKSGIYTIYINNMPEPKKVFCNMDVNGGGWTVIQHREDGSLDFQRGWKEYKMGFGNPSGEYWLGNEFIF
AITSQRQYMLRIELMDWEGNRAYSQYDRFHIGNEKQNYRLYLKGHTGTAGKQSSLILHGADFSTKDADNDNCMCKCALM
LTGGWWFDACGPSNLNGMFYTAGQNHGKLNGIKWHYFKGPSYSLRSTTMMIRPLDF
SEQ ID NO.: 0017: IL2SP-H6-EK-c4bp-Ang1
MYRMQLLSCIALSLALVTNSEHHHHHHGGDDDDKETPEGCEQVLTGKRLMQCLPNPEDVKMALEVYKLSLEIEQLELQR
DSARQSTLDKELGGGGSKPFRDCADVYQAGFNKSGIYTIYINNMPEPKKVFCNMDVNGGGWTVIQHREDGSLDFQRGWK
EYKMGFGNPSGEYWLGNEFIFAITSQRQYMLRIELMDWEGNRAYSQYDRFHIGNEKQNYRLYLKGHTGTAGKQSSLILH
GADFSTKDADNDNCMCKCALMLTGGWWFDACGPSNLNGMFYTAGQNHGKLNGIKWHYFKGPSYSLRSTTMMIRPLDF
SEQ ID NO.: 0018: IL2SP-Ang1-c4bp
MYRMQLLSCIALSLALVTNSEKPFRDCADVYQAGFNKSGIYTIYINNMPEPKKVFCNMDVNGGGWTVIQHREDGSLDFQ
RGWKEYKMGFGNPSGEYWLGNEFIFAITSQRQYMLRIELMDWEGNRAYSQYDRFHIGNEKQNYRLYLKGHTGTAGKQSS
LILHGADFSTKDADNDNCMCKCALMLTGGWWFDACGPSNLNGMFYTAGQNHGKLNGIKWHYFKGPSYSLRSTTMMIRPL
DFGGGGSETPEGCEQVLTGKRLMQCLPNPEDVKMALE
SEQ ID NO.: 0019: CD33SP-c4bp-Ang1-H6
MPLLLLLPLLWAGALAETPEGCEQVLTGKRLMQCLPNPEDVKMALEVYKLSLEIEQLELQRDSARQSTLDKELGGGGSK
PFRDCADVYQAGFNKSGIYTIYINNMPEPKKVFCNMDVNGGGWTVIQHREDGSLDFQRGWKEYKMGFGNPSGEYWLGNE
FIFAITSQRQYMLRIELMDWEGNRAYSQYDRFHIGNEKQNYRLYLKGHTGTAGKQSSLILHGADFSTKDADNDNCMCKC
ALMLTGGWWFDACGPSNLNGMFYTAGQNHGKLNGIKWHYFKGPSYSLRSTTMMIRPLDFHHHHHH
SEQ ID NO.: 0020: Ang1-c4bp
EKPFRDCADVYQAGFNKSGIYTIYINNMPEPKKVFCNMDVNGGGWTVIQHREDGSLDFQRGWKEYKMGFGNPSGEYWLG
NEFIFAITSQRQYMLRIELMDWEGNRAYSQYDRFHIGNEKQNYRLYLKGHTGTAGKQSSLILHGADFSTKDADNDNCMC
KCALMLTGGWWFDACGPSNLNGMFYTAGQNHGKLNGIKWHYFKGPSYSLRSTTMMIRPLDFGGGGSETPEGCEQVLTGK
RLMQCLPNPEDVKMALE
SEQ ID NO.: 0028: c4bp-Ang1
ETPEGCEQVLTGKRLMQCLPNPEDVKMALEVYKLSLEIEQLELQRDSARQSTLDKELGGGGSKPFRDCADVYQAGFNKS
GIYTIYINNMPEPKKVFCNMDVNGGGWTVIQHREDGSLDFQRGWKEYKMGFGNPSGEYWLGNEFIFAITSQRQYMLRIE
LMDWEGNRAYSQYDRFHIGNEKQNYRLYLKGHTGTAGKQSSLILHGADFSTKDADNDNCMCKCALMLTGGWWFDACGPS
NLNGMFYTAGQNHGKLNGIKWHYFKGPSYSLRSTTMMIRPLDF
SEQ ID NO.: 0029: c4bp-Ang2-H6
ETPEGCEQVLTGKRLMQCLPNPEDVKMALEVYKLSLEIEQLELQRDSARQSTLDKELGGGGSISFRDCAEVFKSGHTTN
GIYTLTFPNSTEEIKAYCDMEAGGGGWTIIQRREDGSVDFQRTWKEYKVGFGNPSGEYWLGNEFVSQLTNQQRYVLKIH
LKDWEGNEAYSLYEHFYLSSEELNYRIHLKGLTGTAGKISSISQPGNDFSTKDGDNDKCICKCSQMLTGGWWFDACGPS
NLNGMYYPQRQNTNKFNGIKWYYWKGSGYSLKATTMMIRPADF

In one embodiment, the disclosure provides a polypeptide that comprises a sequence that is at least 90%, 91%, 92%, 93%, 94%, 95%, 96%, 97%, 98%, 99%, or 100% identical to any one of the above sequences. In one embodiment, the polypeptide competes with at least one of the ANG1-C4BP or C4BP-ANG1 described herein for binding to Tie-2 in vitro and/or in vivo. In one embodiment, the polypeptide binds Tie-2 with an affinity of about 100 μM or less, about 50 μM or less, about 25 μM or less, or about 10 μM or less; more preferably have high affinity of about 1 M or less, about 100 nM or less, about 50 nM or less, about 25 nM or less.; preferably binding affinity in the range of about 1 nM to about 10 nM; about 10 nM to about 20 nM; about 20 nM to about 30 nM; about 30 nM to about 40 nM; about 40 nM to about 50 nM; about 50 nM to about 60 nM; about 60 nM to about 70 nM; about 70 nM to about 80 nM; about 80 nM to about 90 nM; or about 90 nM to about 100 nM.

In one embodiment, the polypeptide is used for detection. In one embodiment, the polypeptide is conjugated to a label. In one embodiment, the label is a radioactivity label or a fluorescent label.

Nucleic Acids, Vectors, and Cells

In one embodiment, the disclosure provides nucleic acids encoding the polypeptides of the disclosure. In one embodiment, the nucleic acids comprise one or more of the following sequences:

SEQ ID NO.: 0021: DNA for IL2SP-Ang1-c4bp-H6 [matches both 0008
(no SP) and 0009 (IL2SP)]
ATGTACAGGATGCAACTCCTGTCTTGCATTGCACTAAGTCTTGCACTTGTC
ACGAATTCGAAACCATTTAGAGACTGTGCAGATGTATATCAAGCTGGTTT
TAATAAAAGTGGAATCTACACTATTTATATTAATAATATGCCAGAACCCA
AAAAGGTGTTTTGCAATATGGATGTCAATGGGGGAGGTTGGACTGTAATA
CAACATCGTGAAGATGGAAGTCTAGATTTCCAAAGAGGCTGGAAGGAAT
ATAAAATGGGTTTTGGAAATCCCTCCGGTGAATATTGGCTGGGGAATGAG
TTTATTTTTGCCATTACCAGTCAGAGGCAGTACATGCTAAGAATTGAGTTA
ATGGACTGGGAAGGGAACCGAGCCTATTCACAGTATGACAGATTCCACAT
AGGAAATGAAAAGCAAAACTATAGGTTGTATTTAAAAGGTCACACTGGG
ACAGCAGGAAAACAGAGCAGCCTGATCTTACACGGTGCTGATTTCAGCAC
TAAAGATGCTGATAATGACAACTGTATGTGCAAATGTGCCCTCATGTTAA
CAGGAGGATGGTGGTTTGATGCTTGTGGCCCCTCCAATCTAAATGGAATG
TTCTATACTGCGGGACAAAACCATGGAAAACTGAATGGGATAAAGTGGC
ACTACTTCAAAGGGCCCAGTTACTCCTTACGTTCCACAACTATGATGATTC
GACCTTTAGATTTTGGTGGCGGTGGCTCAGAGACCCCCGAAGGCTGTGAA
CAAGTGCTCACAGGCAAAAGACTCATGCAGTGTCTCCCAAACCCAGAGG
ATGTGAAAATGGCCCTGGAGGTATATAAGCTGTCTCTGGAAATTGAACAA
CTGGAACTACAAAGGGACAGCGCAAGACAATCCACTTTGGATAAAGAAC
TACATCACCATCACCATCACTAA
SEQ ID NO.: 0022: DNA for IL2SP-c4bp-Ang1-H6 [matches both 0010
(no SP) and 0011 (IL2SP)]
ATGTACAGAATGCAGCTGCTGTCCTGTATCGCCCTGAGCCTGGCTCTGGTG
ACCAACTCTGAGACACCAGAGGGATGTGAGCAGGTGCTGACCGGCAAGC
GCCTGATGCAGTGCCTGCCCAATCCTGAGGATGTGAAGATGGCCCTGGAG
GTGTACAAGCTGTCCCTGGAGATCGAGCAGCTGGAGCTGCAGAGGGATTC
CGCCCGGCAGTCTACACTGGACAAGGAGCTGGGAGGAGGAGGCAGCAAG
CCTTTCAGGGATTGTGCCGACGTGTATCAGGCTGGCTTTAACAAGTCTGGC
ATCTACACCATCTATATCAACAATATGCCAGAGCCCAAGAAGGTGTTCTG
CAACATGGACGTGAATGGCGGCGGCTGGACAGTGATCCAGCACAGGGAG
GATGGCAGCCTGGACTTCCAGCGGGGCTGGAAGGAGTACAAGATGGGCT
TTGGCAACCCATCTGGCGAGTATTGGCTGGGCAATGAGTTCATCTTTGCCA
TCACCTCCCAGAGACAGTACATGCTGCGCATCGAGCTGATGGATTGGGAG
GGCAATAGGGCTTACTCTCAGTATGACCGGTTCCATATCGGCAACGAGAA
GCAGAATTACCGGCTGTATCTGAAGGGACACACCGGAACAGCTGGCAAG
CAGTCCAGCCTGATCCTGCATGGCGCCGATTTTTCCACCAAGGACGCTGA
TAACGACAATTGCATGTGCAAGTGCGCCCTGATGCTGACAGGAGGATGGT
GGTTCGACGCTTGCGGACCAAGCAACCTGAATGGCATGTTTTATACAGCT
GGCCAGAACCACGGCAAGCTGAATGGCATCAAGTGGCATTACTTCAAGG
GCCCTTCTTATTCCCTGAGATCCACCACAATGATGATCCGCCCACTGGATT
TTCACCATCACCATCACCATTAA
SEQ ID NO.: 0023: DNA for CD33SP-c4bp-Ang1-H6 [matches both 0010
(no SP) and 0020 (CD33SP)]
ATGCCTCTGCTGCTGCTGCTGCCACTGCTGTGGGCTGGCGCTCTGGCCGAG
ACACCAGAGGGCTGTGAGCAGGTGCTGACAGGCAAGAGACTGATGCAGT
GCCTGCCCAACCCTGAGGATGTGAAGATGGCTCTGGAGGTGTACAAGCTG
TCTCTGGAGATCGAGCAGCTGGAGCTGCAGAGGGATAGCGCCCGGCAGT
CTACCCTGGACAAGGAGCTGGGAGGAGGAGGCTCTAAGCCCTTCCGCGAT
TGTGCTGACGTGTATCAGGCCGGCTTTAATAAGTCCGGCATCTACACCATC
TATATCAACAATATGCCAGAGCCCAAGAAGGTGTTCTGCAACATGGACGT
GAATGGCGGCGGCTGGACAGTGATCCAGCACAGGGAGGATGGCTCCCTG
GACTTCCAGCGGGGCTGGAAGGAGTACAAGATGGGCTTTGGCAACCCTTC
CGGCGAGTATTGGCTGGGCAATGAGTTCATCTTTGCTATCACAAGCCAGA
GACAGTACATGCTGCGCATCGAGCTGATGGATTGGGAGGGCAACAGGGC
CTACAGCCAGTATGACCGGTTCCATATCGGCAACGAGAAGCAGAATTACA
GGCTGTATCTGAAGGGCCACACCGGCACAGCTGGCAAGCAGTCCAGCCTG
ATCCTGCATGGCGCTGACTTCTCCACCAAGGACGCCGATAACGACAATTG
CATGTGCAAGTGCGCTCTGATGCTGACAGGAGGATGGTGGTTCGACGCTT
GTGGACCATCTAACCTGAATGGCATGTTTTATACCGCCGGCCAGAACCAC
GGCAAGCTGAATGGCATCAAGTGGCATTACTTCAAGGGCCCCTCTTATTC
CCTGAGATCCACCACAATGATGATCCGCCCTCTGGATTTTCACCATCACCA
TCACCATTAA
SEQ ID NO.: 0024: DNA for IL2SP-c4bp-Ang2-H6 [matches both 0012
(no SP) and 0013 (IL2SP)]
ATGTACAGAATGCAGCTGCTGAGCTGTATCGCCCTGTCTCTGGCTCTGGTG
ACCAACTCTGAGACACCAGAGGGCTGTGAGCAGGTGCTGACCGGCAAGC
GCCTGATGCAGTGCCTGCCCAATCCTGAGGATGTGAAGATGGCCCTGGAG
GTGTATAAGCTGTCCCTGGAGATCGAGCAGCTGGAGCTGCAGAGAGATTC
TGCTCGCCAGTCCACCCTGGACAAGGAGCTGGGAGGAGGAGGCAGCATC
TCTTTCAGAGATTGTGCCGAGGTGTTTAAGAGCGGCCACACCACAAACGG
CATCTACACCCTGACATTCCCTAATTCTACAGAGGAGATCAAGGCCTATT
GCGACATGGAGGCTGGAGGAGGAGGATGGACCATCATCCAGAGGCGGGA
GGATGGCAGCGTGGACTTCCAGAGGACATGGAAGGAGTACAAAGTGGGC
TTTGGCAACCCATCTGGCGAGTATTGGCTGGGCAACGAGTTCGTGTCCCA
GCTGACCAATCAGCAGCGGTACGTGCTGAAGATCCATCTGAAGGATTGGG
AGGGCAACGAGGCCTACTCTCTGTATGAGCACTTTTACCTGTCCAGCGAG
GAGCTGAATTATCGCATCCATCTGAAGGGCCTGACCGGCACAGCTGGCAA
GATCTCTTCCATCTCCCAGCCCGGCAACGATTTCAGCACCAAGGACGGCG
ATAATGACAAGTGCATCTGTAAGTGCTCCCAGATGCTGACAGGAGGATGG
TGGTTCGACGCTTGCGGACCAAGCAACCTGAATGGCATGTACTATCCCCA
GAGGCAGAACACAAATAAGTTTAATGGCATCAAGTGGTACTATTGGAAG
GGCTCCGGCTATAGCCTGAAGGCCACCACAATGATGATCCGGCCTGCTGA
CTTTCACCATCACCATCACCATTAA
SEQ ID NO.: 0025: IL2SP-H6-EK-Ang1-c4bp [matches both 0014 (no
SP) and 0015 (IL2SP)]
ATGTACAGAATGCAGCTGCTGTCCTGTATCGCCCTGAGCCTGGCTCTGGTG
ACCAACTCTGAGCACCATCACCATCACCATGGCGACGATGACGATAAGAA
GCCATTCCGCGATTGTGCCGACGTGTATCAGGCTGGCTTTAATAAGTCCG
GCATCTACACCATCTATATCAACAATATGCCCGAGCCTAAGAAGGTGTTC
TGCAACATGGATGTGAATGGCGGCGGCTGGACAGTGATCCAGCACAGGG
AGGATGGCAGCCTGGACTTCCAGCGGGGCTGGAAGGAGTACAAGATGGG
CTTTGGCAACCCCTCTGGCGAGTATTGGCTGGGCAATGAGTTCATCTTTGC
CATCACATCCCAGAGACAGTACATGCTGCGCATCGAGCTGATGGATTGGG
AGGGCAACAGGGCTTACTCTCAGTATGACCGGTTCCATATCGGCAACGAG
AAGCAGAATTACAGGCTGTATCTGAAGGGACACACCGGAACAGCTGGCA
AGCAGTCCAGCCTGATCCTGCATGGCGCCGATTTTTCCACCAAGGACGCT
GATAACGACAATTGCATGTGCAAGTGCGCCCTGATGCTGACAGGAGGATG
GTGGTTCGACGCTTGCGGACCAAGCAACCTGAATGGCATGTTTTACACCG
CTGGCCAGAACCACGGCAAGCTGAATGGCATCAAGTGGCATTACTTCAAG
GGCCCTTCTTATTCCCTGAGAAGCACCACAATGATGATCAGGCCTCTGGA
TTTTGGAGGAGGAGGCTCTGAGACACCAGAGGGATGTGAGCAGGTGCTG
ACAGGCAAGCGGCTGATGCAGTGCCTGCCAAATCCCGAGGACGTGAAGA
TGGCCCTGGAGGTGTATAAGCTGTCCCTGGAGATCGAGCAGCTGGAGCTG
CAGAGGGATTCCGCCCGGCAGTCTACACTGGACAAGGAGCTGTAA
SEQ ID NO.: 0026: DNA for IL2SP-H6-EK-c4bp-Ang1 [matches both
0016 (no SP) and 0017 (IL2SP)]
ATGTACAGAATGCAGCTGCTGTCCTGTATCGCCCTGAGCCTGGCTCTGGTG
ACCAACTCTGAGCACCATCACCATCACCATGGCGGCGACGATGACGATAA
GGAGACACCCGAGGGCTGTGAGCAGGTGCTGACAGGCAAGCGCCTGATG
CAGTGCCTGCCCAATCCTGAGGATGTGAAGATGGCCCTGGAGGTGTACAA
GCTGTCCCTGGAGATCGAGCAGCTGGAGCTGCAGAGGGATTCCGCCCGGC
AGTCTACACTGGACAAGGAGCTGGGAGGAGGAGGCAGCAAGCCTTTCAG
GGATTGTGCCGACGTGTATCAGGCTGGCTTTAACAAGTCTGGCATCTACA
CCATCTATATCAACAATATGCCAGAGCCCAAGAAGGTGTTCTGCAACATG
GACGTGAATGGCGGCGGCTGGACAGTGATCCAGCACAGGGAGGATGGCA
GCCTGGACTTCCAGCGGGGCTGGAAGGAGTACAAGATGGGCTTTGGCAAC
CCATCTGGCGAGTATTGGCTGGGCAATGAGTTCATCTTTGCCATCACCTCC
CAGAGACAGTACATGCTGCGCATCGAGCTGATGGATTGGGAGGGCAATA
GGGCTTACTCTCAGTATGACCGGTTCCATATCGGCAACGAGAAGCAGAAT
TACCGGCTGTATCTGAAGGGACACACCGGAACAGCTGGCAAGCAGTCCA
GCCTGATCCTGCATGGCGCCGATTTTTCCACCAAGGACGCTGATAACGAC
AATTGCATGTGCAAGTGCGCCCTGATGCTGACAGGAGGATGGTGGTTCGA
CGCTTGCGGACCAAGCAACCTGAATGGCATGTTTTATACAGCTGGCCAGA
ACCACGGCAAGCTGAATGGCATCAAGTGGCATTACTTCAAGGGCCCTTCT
TATTCCCTGAGATCCACCACAATGATGATCCGCCCACTGGATTTTTAA
SEQ ID NO.: 0027: DNA for IL2SP-Ang1-c4bp (tag-less)
[matches 0018]
ATGTACAGAATGCAGCTGCTGTCCTGTATCGCCCTGAGCCTGGCTCTGGTG
ACCAACTCTGAGAAGCCATTCCGCGATTGTGCCGACGTGTATCAGGCTGG
CTTTAATAAGTCCGGCATCTACACCATCTATATCAACAATATGCCCGAGC
CTAAGAAGGTGTTCTGCAACATGGATGTGAATGGCGGCGGCTGGACAGTG
ATCCAGCACAGGGAGGATGGCAGCCTGGACTTCCAGCGGGGCTGGAAGG
AGTACAAGATGGGCTTTGGCAACCCCTCTGGCGAGTATTGGCTGGGCAAT
GAGTTCATCTTTGCCATCACATCCCAGAGACAGTACATGCTGCGCATCGA
GCTGATGGATTGGGAGGGCAACAGGGCTTACTCTCAGTATGACCGGTTCC
ATATCGGCAACGAGAAGCAGAATTACAGGCTGTATCTGAAGGGACACAC
CGGAACAGCTGGCAAGCAGTCCAGCCTGATCCTGCATGGCGCCGATTTTT
CCACCAAGGACGCTGATAACGACAATTGCATGTGCAAGTGCGCCCTGATG
CTGACAGGAGGATGGTGGTTCGACGCTTGCGGACCAAGCAACCTGAATGG
CATGTTTTACACCGCTGGCCAGAACCACGGCAAGCTGAATGGCATCAAGT
GGCATTACTTCAAGGGCCCTTCTTATTCCCTGAGAAGCACCACAATGATG
ATCAGGCCTCTGGATTTTGGAGGAGGAGGCTCTGAGACACCAGAGGGATG
TGAGCAGGTGCTGACAGGCAAGCGGCTGATGCAGTGCCTGCCAAATCCCG
AGGACGTGAAGATGGCCCTGGAGGTGTATAAGCTGTCCCTGGAGATCGAG
CAGCTGGAGCTGCAGAGGGATTCCGCCCGGCAGTCTACACTGGACAAGG
AGCTGTAA

In one embodiment, the disclosure provides a nucleic acid that comprises a sequence that is at least 90%, 91%, 92%, 93%, 94%, 95%, 96%, 97%, 98%, 99%, or 100% identical to any one of the above sequences. In one embodiment, the nucleic acid sequence is codon-optimized.

In one embodiment, the disclosure provides a vector comprising one or more of the nucleic acid sequences of the disclosure. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., lentiviruses, retroviruses, replication defective retroviruses, adenoviruses and adeno-associated viruses, herpes virus), which serve equivalent functions. The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. In some embodiments, the lentiviral vector is a human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); or simian immunodeficiency virus (SIV) vector. Other means of genetically modifying cells to express the spFv molecules of the disclosure include transposase enzymes, mRNA transfection, non-integrative lentivirus, “Sleeping Beauty (SB)” transposons, endonuclease enzymes, in situ transfection with DNA nanocarriers.

In some embodiments, the vector is an adenoviral vector, an adenovirus-associated vector, a DNA vector, a lentiviral vector, a plasmid, a retroviral vector, or an RNA vector. In some embodiments, the vector is a viral vector. In some embodiments, the vector is a retroviral vector. In some embodiments, the vector is a lentiviral vector.

In one embodiment, the disclosure provides a host cell comprising a polypeptide of the disclosure. In one embodiment, the disclosure provides a host cell comprising a nucleic acid of the disclosure.

In one embodiment, the disclosure provides a host cell comprising a vector of the disclosure. Examples of host cells are provided elsewhere in the specification.

Compositions

In one aspect, the present disclosure provides a composition comprising a polypeptide disclosed herein. In one aspect, the disclosure provides a nucleic acid described herein. In one aspect, the present disclosure provides a composition comprising a vector described. In one aspect, the present disclosure provides a composition comprising a host cell described herein.

In one embodiment, the compositions are pharmaceutical compositions, comprising a polynucleotide described herein, a vector described herein, a polypeptide described herein, or an host cell described herein. In some embodiments, the composition comprises a pharmaceutically acceptable carrier, diluent, solubilizer, emulsifier, preservative, and/or adjuvant.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant excipient, or vehicle with which the therapeutic is administered. Such pharmaceutical carriers may be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions may also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, sodium phosphate, sodium acetate, L-Histidine, dried skim milk, glycerol, propylene, glycol, water, ethanol and the like. The composition, if desired, may also contain minor amounts of wetting or emulsifying agents, or pH buffering agents. These compositions may take the form of solutions, suspensions, emulsion, tablets, pills, capsules, powders, sustained-release formulations and the like. Generally, the ingredients of compositions of the disclosure are supplied either separately or mixed together in unit dosage form, for example, for the vector and polypeptide-based compositions, as a dry lyophilized powder or water free concentrate in a hermetically sealed container such as an ampoule or sachet indicating the quantity of active agent. Where the composition is to be administered by infusion (e.g., host cell compositions), it may be dispensed with an infusion bottle containing sterile pharmaceutical grade water or saline. Where the composition is administered by injection, an ampoule of sterile water for injection or saline may be provided so that the ingredients may be mixed prior to administration.

The compositions of the disclosure include bulk drug compositions useful in the manufacture of pharmaceutical compositions (e.g., impure or non-sterile compositions) and pharmaceutical compositions (i.e., compositions that are suitable for administration to a subject or patient) which may be used in the preparation of unit dosage forms. Such compositions comprise a prophylactically or therapeutically effective amount of the prophylactic and/or therapeutic dual specificity polypeptide molecule (agent) disclosed herein or a combination of the agent and a pharmaceutically acceptable carrier. Preferably, compositions of the disclosure comprise a prophylactically or therapeutically effective amount of one or more molecules of the disclosure and a pharmaceutically acceptable carrier. The pharmaceutical compositions preferably comprise the molecules either in the free form or as a salt. Preferably, the salts are pharmaceutical acceptable salts of the molecules, such as, for example, the chloride or acetate (trifluoroacetate) salts. It has to be noted that the salts of the molecules according to the present disclosure differ substantially from the molecules in their state(s) in vivo, as the molecules are not salts in vivo. In an aspect, the aqueous carrier contains multiple components, such as water together with a non-water carrier component, such as those components described herein. In another aspect, the aqueous carrier is capable of imparting improved properties when combined with a peptide or other molecule described herein, for example, improved solubility, efficacy, and/or improved immunotherapy. In addition, the composition may contain excipients, such as buffers, binding agents, blasting agents, diluents, flavors, lubricants, etc. A “pharmaceutically acceptable diluent,” for example, may include solvents, bulking agents, stabilizing agents, dispersion media, coatings, antibacterial and antifungal agents, isotonic and absorption delaying agents, and the like which are physiologically compatible. Examples of pharmaceutically acceptable diluents include one or more of saline, phosphate buffered saline, dextrose, glycerol, ethanol, and the like as well as combinations thereof. In many cases it will be preferable to include one or more isotonic agents, for example, sugars such as trehalose and sucrose, polyalcohols such as mannitol, sorbitol, or sodium chloride in the composition. Pharmaceutically acceptable substances such as wetting or minor amounts of auxiliary substances such as wetting or emulsifying agents, preservatives or buffers, are also within the scope of the present disclosure. In addition, the composition may contain excipients, such as buffers, binding agents, blasting agents, diluents, flavors, and lubricants.

In an aspect, peptides or other molecules described herein may be combined with an aqueous carrier. In an aspect, the aqueous carrier is selected from ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, salts or electrolytes, such as protamine sulfate, disodium hydrogen phosphate, dicalcium phosphate, potassium hydrogen phosphate, sodium chloride, zinc salts, colloidal silica, magnesium trisilicate, polyvinyl pyrrolidone, polyvinylpyrrolidone-vinyl acetate, cellulose-based substances (e.g., microcrystalline cellulose, hydroxypropyl methylcellulose, hydroxypropyl methylcellulose acetate succinate, hydroxypropyl methylcellulose Phthalate), starch, lactose monohydrate, mannitol, trehalose sodium lauryl sulfate, and crosscarmellose sodium, polyethylene glycol, sodium carboxymethylcellulose, polyacrylates, polymethacrylate, waxes, polyethylene-polyoxypropylene-block polymers, polyethylene glycol and wool fat.

In other embodiments, the composition is selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In certain embodiments, the vehicle for parenteral injection is sterile distilled water in which composition described herein, with or without at least one additional therapeutic agent, is formulated as a sterile, isotonic solution, properly preserved. In certain embodiments, the preparation involves the formulation of the desired molecule with polymeric compounds (such as polylactic acid or polyglycolic acid), beads or liposomes, that provide for the controlled or sustained release of the product, which are then be delivered via a depot injection. In certain embodiments, implantable drug delivery devices are used to introduce the desired molecule.

The pH of the composition generally should not be equal to the isoelectric point of the particular chimeric polypeptides of the disclosure and may range from about 4.0 to about 7.0, about 5.0 to about 6.0, or about 5.5 to about 6.0. In certain embodiments, the composition or formulation of the present disclosure has a pH of about 5.5, 5.6, 5.7, 5.8, 5.9, or 6.0. Buffering agents may help to maintain the pH of the compositions of the disclosure in the range which approximates physiological conditions. They may be present at concentration ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris may be used.

Preservatives may be added to retard microbial growth and may be added in amounts ranging from 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” may be added to ensure isotonicity of liquid compositions of the present disclosure and include polhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers may be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, .alpha.-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilizers may be present in the range from 0.1 to 10,000 weights per part of weight active protein.

Non-ionic surfactants or detergents (also known as “wetting agents”) may be added to help solubilize the therapeutic agent as well as to protect the Ang1-containing molecule against agitation-induced aggregation, which also permits the formulation to be exposed to shear surface stressed without causing denaturation of the protein. Suitable non-ionic surfactants include polysorbates (20, 80, and others), polyoxamers (184, 188 and others), Pluronic polyols, polyoxyethylene sorbitan monoethers (TWEEN-20, TWEEN-80 and others). Nonionic surfactants may be present in a range of about 0.05 mg/mL to about 1.0 mg/mL, for example about 0.07 mg/mL to about 0.2 mg/mL.

Also provided are methods for engineering, preparing, and producing the cells, compositions containing the cells, and kits and devices containing and for using, producing and administering the cells. Any of the compositions described herein may be comprised in a kit. The kit components are provided in suitable container means.

Methods of Use

In one embodiment, the disclosure provides that recombinantly produced ANG1-C4BP and C4BP-ANG1 potently activate Tie2 in vitro, in vivo, in human cells and/or mouse models.

In one embodiment, the disclosure provides methods of decreasing or inhibiting vascular leakage or plasma permeability. In one embodiment, the disclosure provides methods of promoting growth and maintaining endothelial structural integrity of vasculature.

In one embodiment, the intended indications of therapeutic use of ANG1-C4BP series of biologics includes vascular eye diseases, such as primary open angle glaucoma caused by defects in limbus capillary plexus or Schlemm's canal drainage system, and types of primary or secondary retinopathy, as well as for systemic treatment of vascular leakage as in cancer neoangiogenesis, conditions of inflammation, among others. In some embodiments, the chimeric polypeptides of the disclosure are more biologically active than any other Angiopoietin-related biologic described to date, including Bow-Ang1 and COMP:Ang1 because of its unexpected advantageous properties.

In one embodiment, the disclosure provides a method of reducing vascular permeability or leakage in a subject in need thereof comprising administering to the subject an effective amount of a polypeptide of the disclosure, a cell of the disclosure, a nucleic of the disclosure, a vector of the disclosure, a protein complex of the disclosure, and/or a pharmaceutical composition of the disclosure. In one embodiment, the vascular permeability or leakage has been increased in the skin, eye, lung, kidney, brain, liver, heart, and intestine. In one embodiment, the vascular permeability or leakage has been increased in response to increased levels of an agent selected from VEGF, chemical agents including toxic gas, infectious bacteria and viruses, autoimmune antibodies, and antibody drugs that cause endothelium dysfunction and vascular damage.

In one embodiment, the disclosure provides a method of treating a disease or disorder accompanied by abnormal vascular permeability or leakage in a subject in need thereof comprising administering to the subject an effective amount of a polypeptide of the disclosure, a cell of the disclosure, a nucleic of the disclosure, a vector of the disclosure, a protein complex of the disclosure, and/or a pharmaceutical composition of the disclosure.

In one embodiment, the disclosure provides a method of treating a disease or disorder that responds to Tie2 activation in a subject in need thereof comprising administering to the subject an effective amount of a polypeptide of the disclosure, a cell of the disclosure, a nucleic of the disclosure, a vector of the disclosure, a protein complex of the disclosure, and/or a pharmaceutical composition of the disclosure. In one embodiment, a disease or disorder that responds to Tie2 activation is any disease or disorder wherein at least one sign or the severity of a symptom, the frequency with which such a symptom is experienced by a patient, or both, is reduced or eliminated by Tie2 activation.

In one embodiment, the disorder is selected from cancer in tumor angiogenesis and metastasis, ocular diseases or disorders such as glaucoma, bacterial sepsis, severe viral infections, protozoan infections such as falciparum malaria, inflammation, lethal anthrax, chronic kidney disease, acute kidney injury and renal dysfunction, acute lung injury and bronchial dysfunction, acute respiratory distress syndrome, obstructive lung disease, acute liver failure, acute pancreatitis, stroke, myocardial infarction, congestive heart failure, amyotrophic lateral sclerosis, Alzheimer's disease, Huntington's disease, Parkinson's disease, peripheral neuropathies, diabetic nephropathy and retinopathy, wound healing, arthritis, fibrotic conditions, ischemia-reperfusion injury, traumatic brain injury, epilepsy, multiple sclerosis, organ transplantation and allograft rejection.

In one embodiment, the cancer is selected from any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. In one embodiment, treatment with the compounds of the disclosure is combined with other cancer therapies including, but not limited to, chemotherapy and radiation.

In one embodiment, the disclosure is directed to a method of treating an angiogenesis-mediated disease in a subject in need thereof. The method comprising administering an effective amount of the composition including any other agents described above. Exemplary angiogenesis-mediated diseases capable of being treated include non-ocular hemorrhage, myocardial infarction, stroke, cancer, atherosclerosis, ischaemic heart disease, coronary heart disease, peripheral arterial disease, wound healing disorders, and the like.

In one embodiment, the ocular disease or disorder is selected from the group consisting of age-related macular degeneration (AMD), macular degeneration, macular edema, diabetic macular edema (DME) (including focal, non-center DME and diffuse, center-involved DME), retinopathy, diabetic retinopathy (DR) (including proliferative DR (PDR), non-proliferative DR (NPDR), and high-altitude DR), other ischemia-related retinopathies, retinopathy of prematurity (ROP), retinal vein occlusion (RVO) (including central (CRVO) and branched (BRVO) forms), CNV (including myopic CNV), corneal neovascularization, a disease associated with corneal neovascularization, retinal neovascularization, a disease associated with retinal/choroidal neovascularization, pathologic myopia, von Hippel-Lindau disease, histoplasmosis of the eye, familial exudative vitreoretinopathy (FEVR), Coats' disease, Norrie Disease, Osteoporosis-Pseudoglioma Syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis (including infectious conjunctivitis and non-infectious (e.g., allergic) conjunctivitis), Leber congenital amaurosis, uveitis (including infectious and non-infectious uveitis), choroiditis, ocular histoplasmosis, blepharitis, dry eye, traumatic eye injury, and Sjögren's disease. In one embodiment, the ocular disease or disorder is glaucoma, AMD, or DME.

In one embodiment, the method further comprises administering a second agent. In one embodiment, the second agent is selected from an antibody, an anti-inflammatory agent, an anti-angiogenic agent, a cytokine, a cytokine antagonist, a corticosteroid, and an analgesic.

In one embodiment, the anti-angiogenic agent includes a compound selected from a VE-PTP inhibitor, bevacizumab, itraconazole, carboxyamidotriazole, TNP-470, CM101, INF-alpha, IL-12, platelet factor-4, suramin, SU5416, thrombospondin, a VEGFR antagonist, an angiostatic steroid plus heparin, Cartilage-Derived Angiogenesis Inhibitory Factor, a matrix metalloproteinase inhibitor, angiostatin, endostatin, 2-methoxyestradiol, tecogalan, tetrathiomolybdate, thalidomide, thrombospondin, prolactin, linomide, αvβ3 inhibitors, ramucirumab, tasquinimod, ranibizumab, sorafenib, sunitinib, pazopanib, and everolimus.

In one embodiment, the anti-angiogenic agent is a VEGF antagonist. In one embodiment, the VEGF antagonist is an anti-VEGF antibody, an anti-VEGF receptor antibody, a soluble VEGF receptor fusion protein, an aptamer (e.g. pegaptanib (MACUGEN®)), an anti-VEGF DARPin® (e.g., abicipar pegol), or a VEGFR tyrosine kinase inhibitor (e.g., 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787), semaxaminib (SU5416), and SUTENT® (sunitinib)). In one embodiment, the anti-VEGF antibody is ranibizumab (LUCENTIS®), RTH-258, or a bispecific anti-VEGF antibody. In one embodiment, the bispecific anti-VEGF antibody is an anti-VEGF/anti-Ang2 antibody. In one embodiment, the anti-VEGF/anti-Ang2 antibody is RG-7716. In one embodiment, the soluble VEGF receptor fusion protein is aflibercept (EYLEA®).

Additional therapeutic agents suitable for use in combination with the compositions and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept,adipotide, denileukin diftitox, mTOR inhibitors such as Everolimus and Temsirolimus, hedgehog inhibitors such as sonidegib and vismodegib, and CDK inhibitors such as CDK inhibitor (palbociclib).

Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

The method may include the further step of determining the efficacy of ANG1-C4BP and its variants in the animal model; and evaluating systemic activation of Tie2, such as in the lung, thereby determining the efficacy of the biologic. The animal used in the methods may be a rodent, or a larger animal such as a rabbit. However, any appropriate animal may serve as an in vivo animal model. In vivo animal models of Tie2 associated diseases or disorders are well known in the art.

### EXAMPLES

### Example 1

### Construct Design and Small-Scale Expression

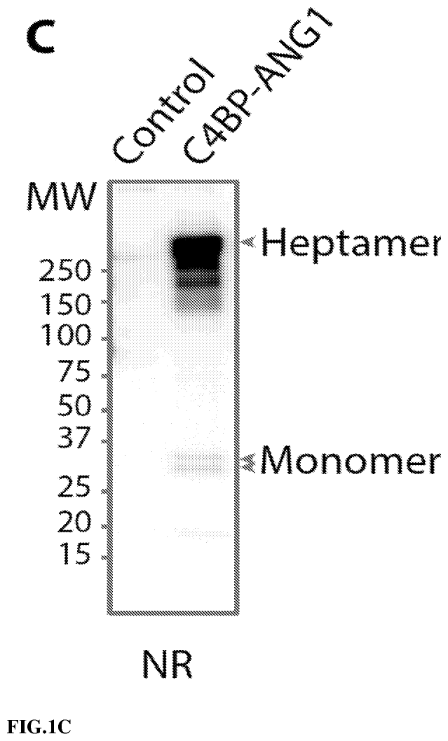

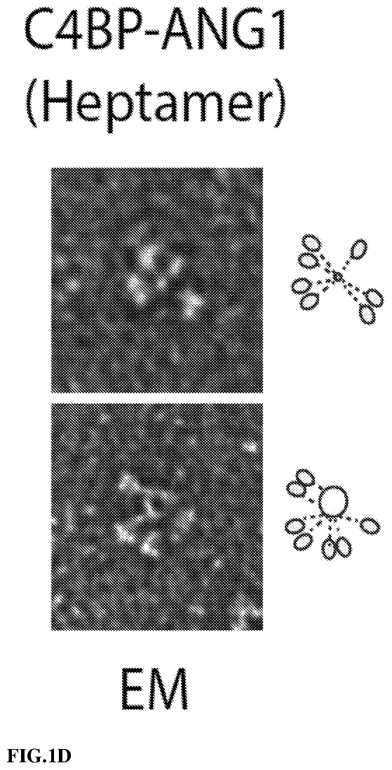

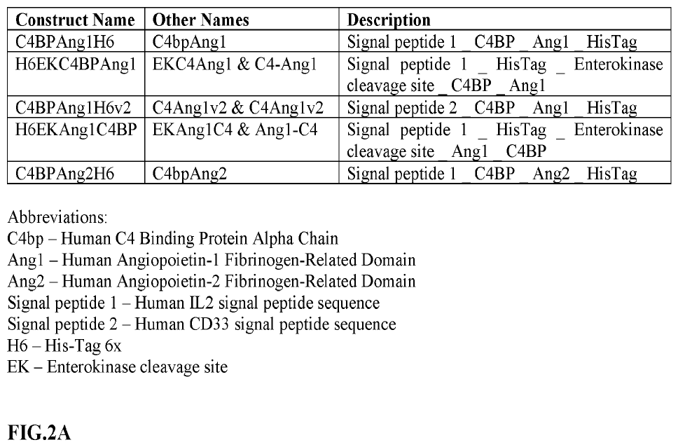

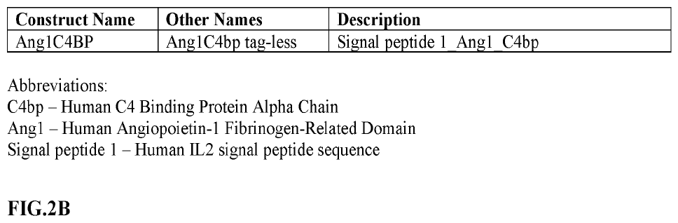

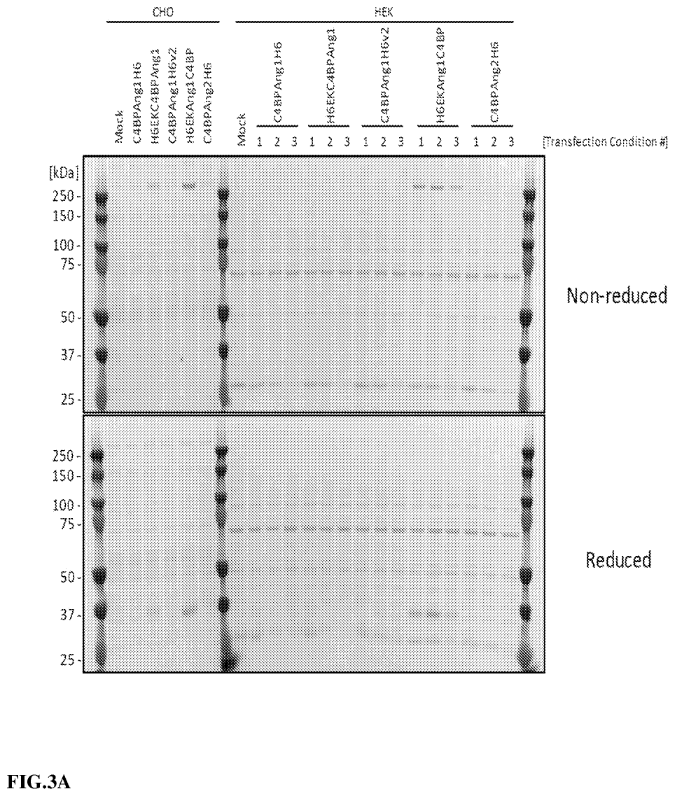

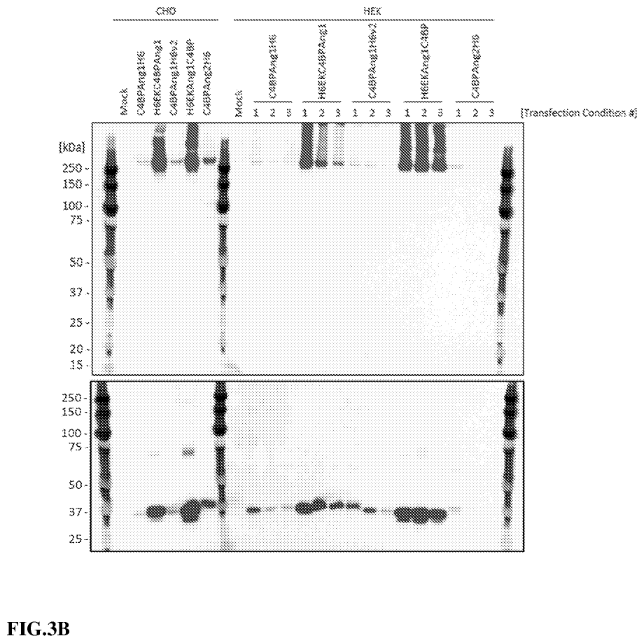

Gene synthesis of cDNAs encoding ANG1-C4BP chimeric fusion constructs (FIG. 2) was performed at GenScript® Corporation. The codon-optimized (CHO codon bias) cDNAs of the constructs were subcloned into pTT81® expression vector or similar, and CHO and HEK293 cells were transiently transfected for small scale production analysis (FIG. 3). Using transient expression different chimeric constructs of ANG1 and ANG2 fused to C4BP were tested. All recombinant fusion proteins were secreted as heptamers of ˜280 kDa, with constructs H6EKC4BPAng1 and H6EKAng1C4BP expressed at highest levels, as shown with Ponceau S solution staining under non-reduced and reduced conditions (FIG. 3A), as well as non-reducing and reducing SDS-PAGE western blots using anti-His-Tag antibody (FIG. 3B). The multimeric state of recombinant fusion proteins was confirmed by comparing the behavior of the protein on an SDS-PAGE gel in the presence and absence of the reducing agent beta-mercaptoethanol.

### Example 2

### Large Scale Expression of Angiopoietin-C4BP and C4BP-Angiopoietin

For stable expression of different ANG1-C4BP constructs Canada's National Research Council (NRC) CHO-BRI (clone 55E1) cells were transfected and selected by addition of methionine sulfoximine (MSX) for approximately two weeks. Pool expression of stable CHO-BRI and fed-batch production in shaker flasks followed. Cultures were agitated on an orbital shaker in a humidified incubator maintained at a desired temperature with a 5% CO₂ overlay. Cells were maintained in chemically defined PowerCHO2 medium, while fed-batch cultures were performed using BalanCD growth A as a basal medium supplemented with MSX and 0.3% pluronic F68. For fed-batch cultures, the feed rate was adjusted daily to maintain a prescribed constant glucose level in the cultures. CHO-BRI is a stable expression system for recombinant protein production that uses the cumate inducible expression platform to generate CHO pools that stably express between 200 and 1000 mg/L in under four weeks post-transfection—two weeks for pool selection and expansion, and two for production (Poulain A, et al. Rapid protein production from stable CHO cell pools using plasmid vector and the cumate gene-switch. J Biotechnol. 2017; 255:16-27).

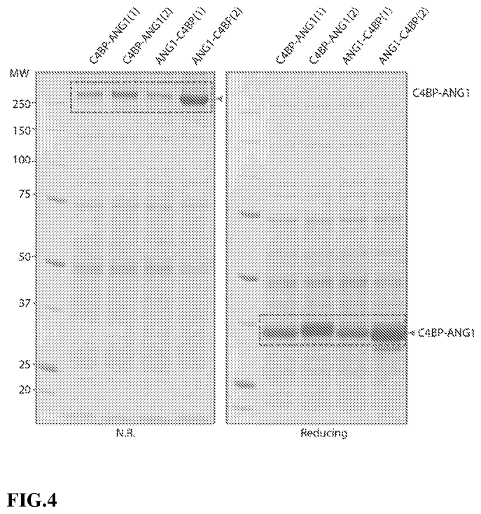

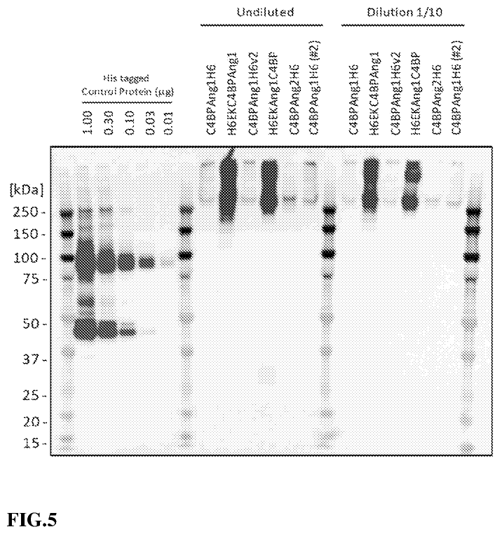

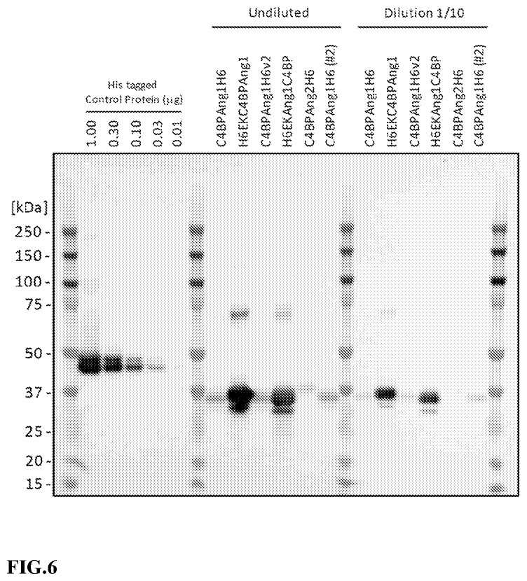

Recombinant protein products of chimeric fusion Angiopoietin-C4BP constructs were found at the expected molecular weight following analysis with SDS-PAGE Coomassie blue stain (FIG. 4), as well as non-reduced (FIG. 5) and reduced (FIG. 6) SDS-PAGE separation and immunoblotting with anti-His-Tag antibody. Therefore, stable CHO expression of ANG1-C4BP and C4BP-ANG1 chimeric fusion proteins shows self-assembly as a predicted heptamer in cell culture medium.

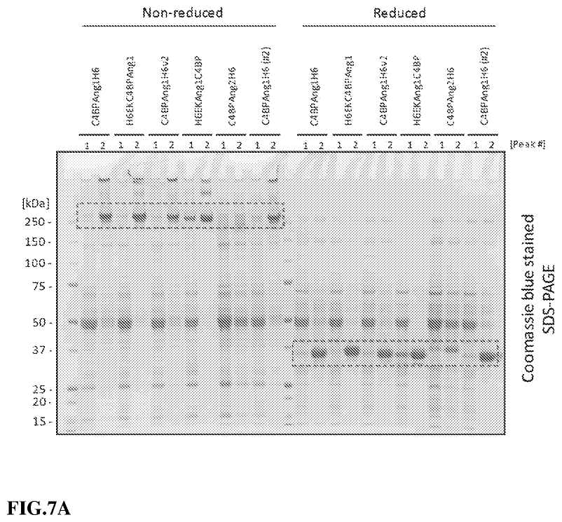

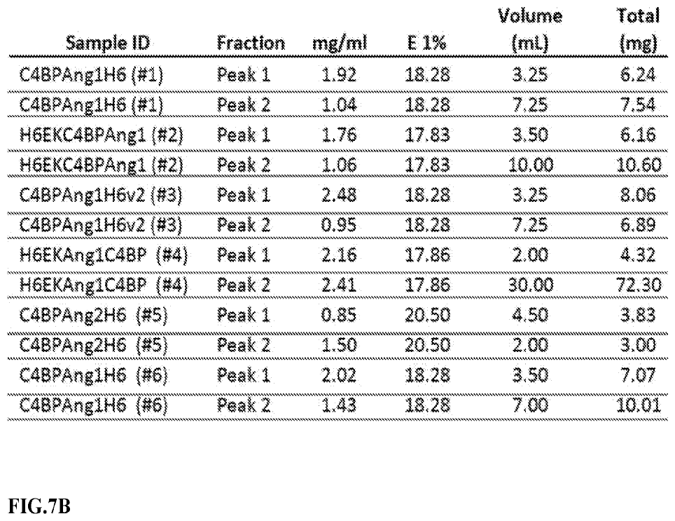

Fed-batch production in shaker flasks was performed to obtain the recombinant proteins, which were harvested and purified by centrifugation and filtration, followed by immobilized metal affinity chromatography (IMAC) purification using gradient for elution, desalting and buffer exchanged into DPBS, concentration, sterile-filtration and quantified by absorbance @ 280 nm. Purified material was further analyzed by UPLC-SEC (ultra-performance liquid chromatography-size exclusion chromatography) to determine aggregation levels and by SDS-PAGE (reduced & non-reduced) for purity determination. The recombinant fusion protein products were found at the right molecular weight in peak #2 fraction (FIG. 7A). Overview of IMAC purified fractions for peak 1 and 2 in terms of volume and total amount for each recombinant fusion protein produced (FIG. 7B).

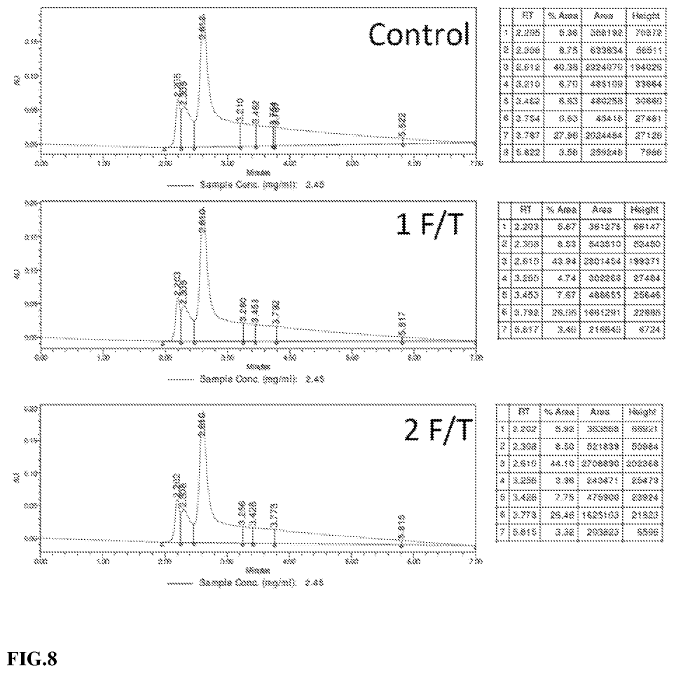

Purified ANG1-C4BP was subjected to frozen storage at −80° C., and up to two rounds of freeze-and-thaw (F/T) cycles to determine protein stability (FIG. 8). No noticeable UPLC-SEC analytical profile changes were observed under these conditions, demonstrating stability.

### Example 3

### In Vitro Biological Activity of ANG1-C4BP and C4BP-ANG1

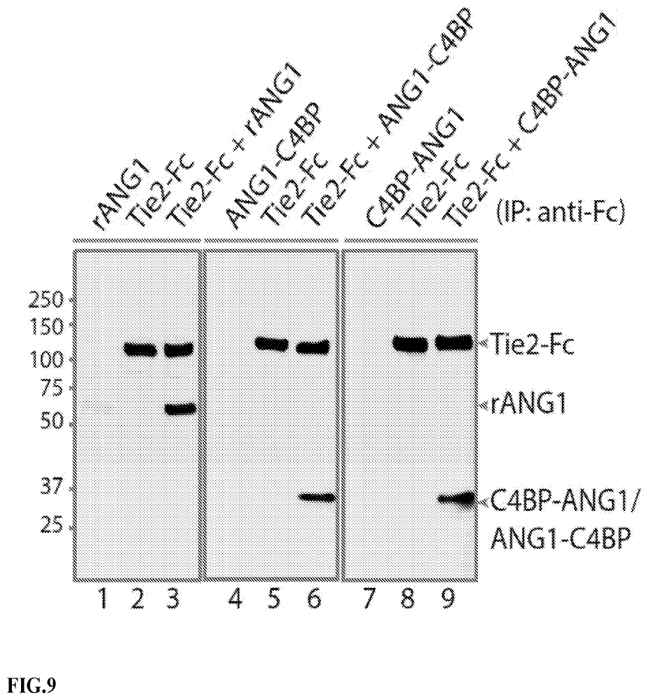

Purified ANG1-C4BP and C4BP-ANG1 were tested for functional binding with the ectodomain of Tie2 in a recombinant fusion with Fc (referred to as Tie2-Fc). Both ANG1-C4BP and C4BP-ANG1 can bind Tie2-Fc (FIG. 9).

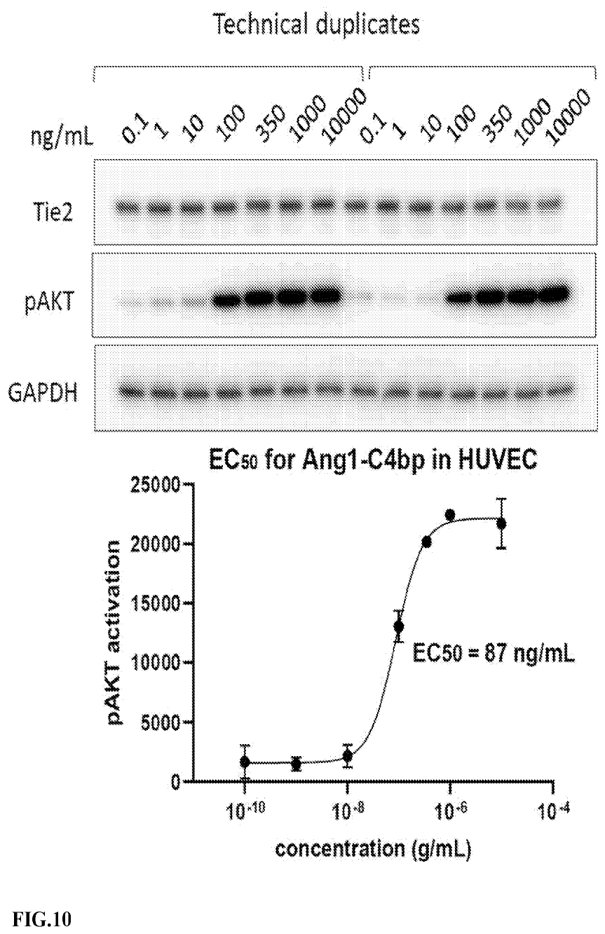

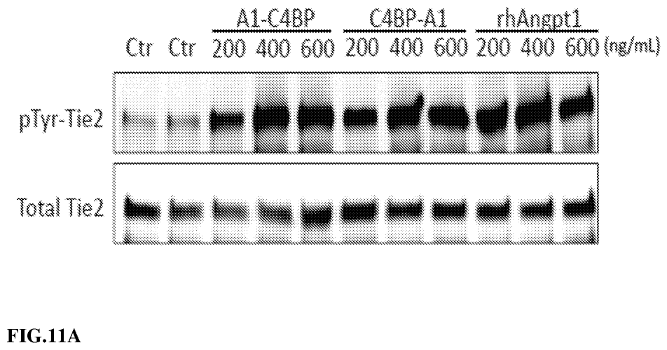

To determine the potency of ANG1-C4BP, the half-maximal effective concentration (EC₅₀) was measured in HUVEC treated for 20 minutes. The phospho-AKT (pAKT) EC₅₀ for ANG1-C4BP was 87 ng/mL (FIG. 10).

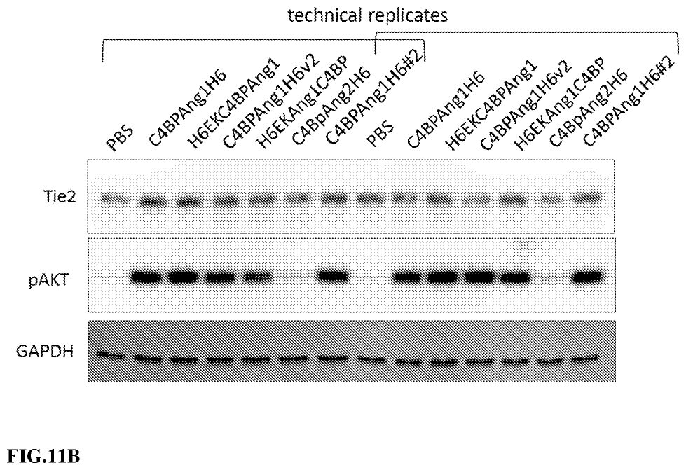

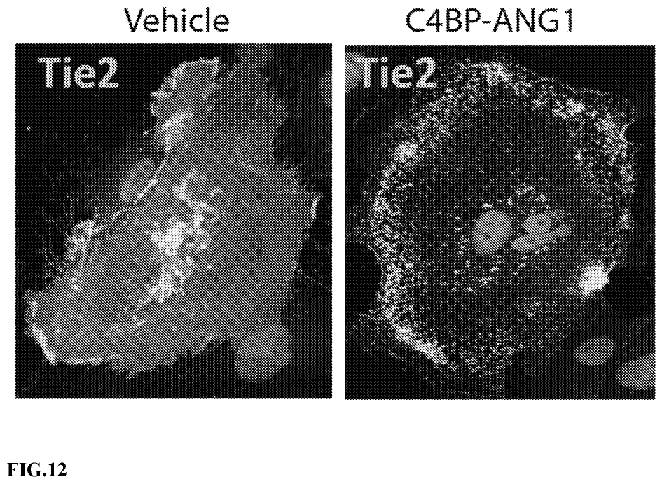

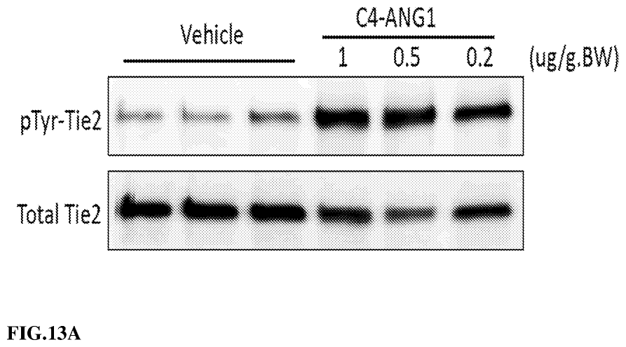

To assess the biological activity and potency, different recombinant protein products obtained from chimeric fusion constructs were used to treat HUVEC at various concentrations for 20 minutes. The recombinant protein products of chimeric fusion constructs between ANG1 and C4BP were effective at activating (phosphorylating) Tie2 receptor tyrosine kinase (FIG. 11A) and inducing phosphorylation of its downstream target AKT (FIG. 111B). The sole exception was the product of chimeric construct made from C4BP fused to Angiopoietin-2 FLD (C4bpAng2H6). At the cellular level, C4BP-ANG1 stimulated Tie2 and reorganized its subcellular distribution in cultured HUVEC. Following C4BP-ANG1 treatment, cell surface Tie2 was clustered and pooled to the junctions (FIG. 12). In summary, ANG1-C4BP and C4BP-ANG1 recombinant fusion proteins in either configuration form stable heptamers that bind to cognate Tie2 receptors resulting in their activation, in keeping with an expected heptavalent clustering effect of ANG1-C4BP variants.

### Example 4

### In Vivo Biological Activity of C4BP-ANG1

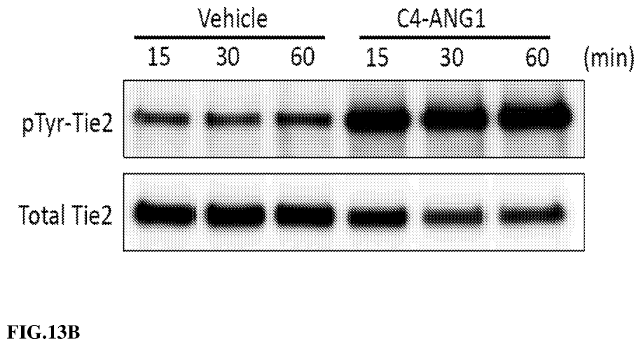

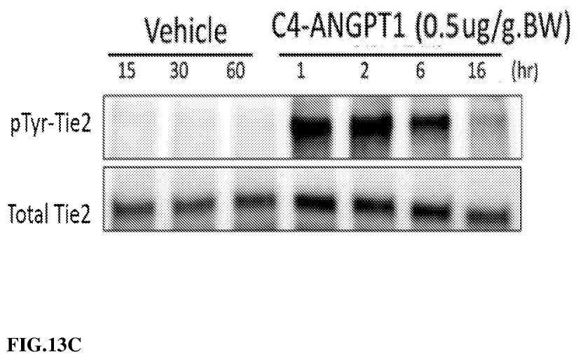

To determine the biological activity of C4BP-ANG1 in vivo, BALB/c mice were intravenously injected with different concentrations ranging from 0.2 to 1 ug/g of body weight (FIG. 13A). The three concentrations used resulted in activation of Tie2 in the lung in a dose dependent manner. C4BP-ANG1 activated Tie2 as soon as 15 minutes (FIG. 13B) and lasted for at least 6 hours post treatment, with lower level activation apparent at 16 hours post treatment (FIG. 13C).

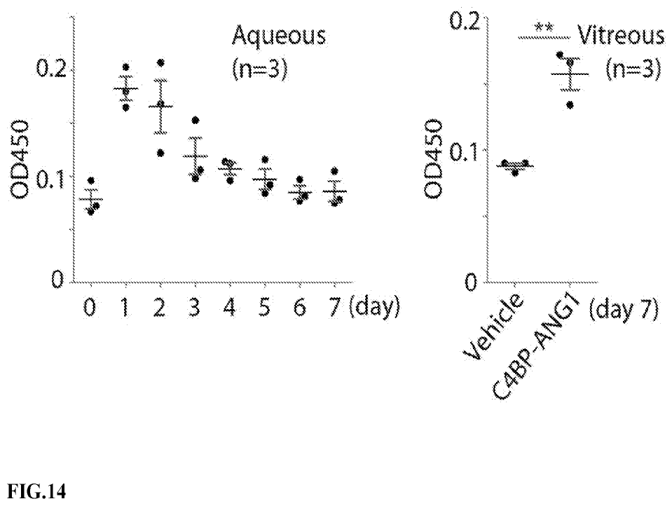

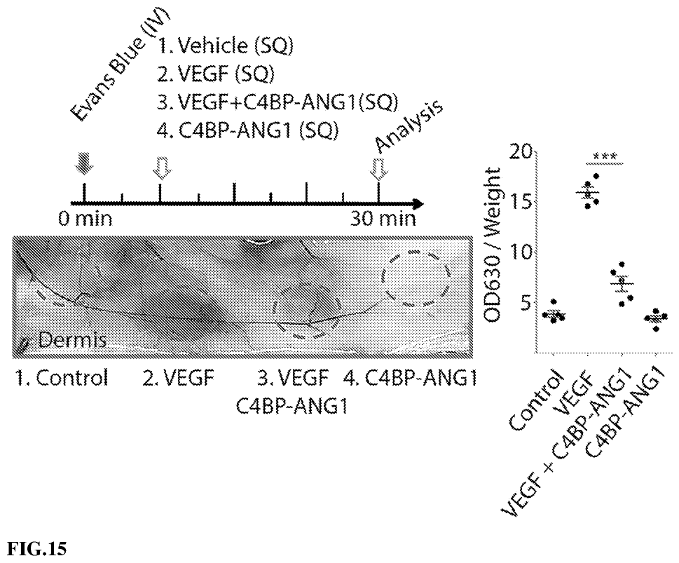

Three white New Zealand rabbits were used in an ocular pharmacokinetic experiment to determine the level of C4BP-ANG1 in aqueous humour following a single intravitreal injection of the recombinant fusion protein. Aqueous humor was collected before intravitreal injections of 100 ug of C4BP-ANG1 into the right eye of each rabbit, and from day 1 until day 7 after the injection, by performing daily aqueous humour tap collections. Vitreous humor was collected after euthanizing the rabbits on day 7. Intravitreal injection in rabbits showed persistent C4BP-ANG1 in aqueous humour (AH) for few days, as measured by ELISA, starting with a spike in the first two to three days and then gradually leveling off to baseline (FIG. 14). A method with greater sensitivity would be required to detect AH levels of C4BP-ANG1 three days following intravitreal injection. The C4BP-ANG1 was detected in the vitreous humour (VH) from right eyes even after seven days post treatment, while the left VH served as a vehicle negative control (FIG. 14).

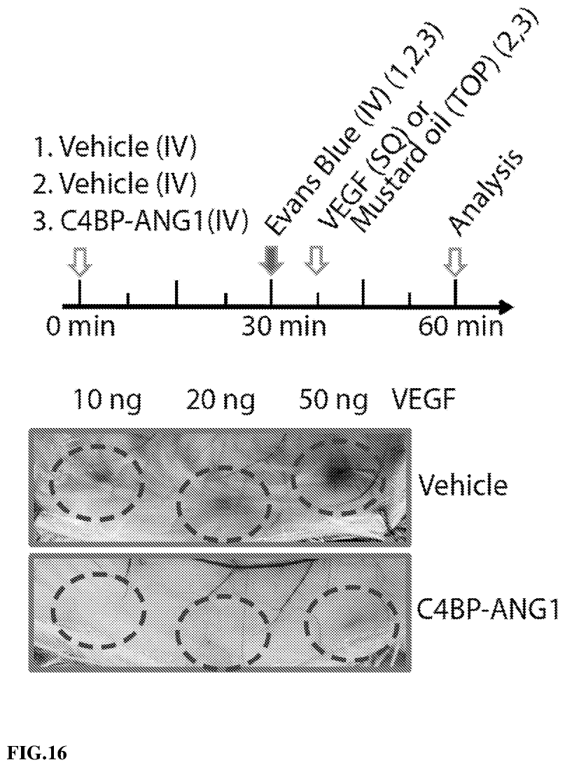

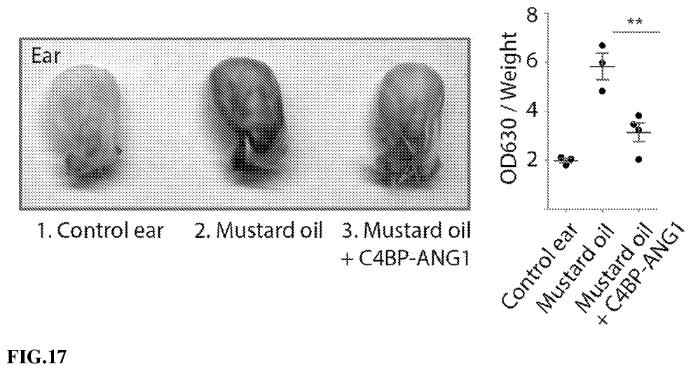

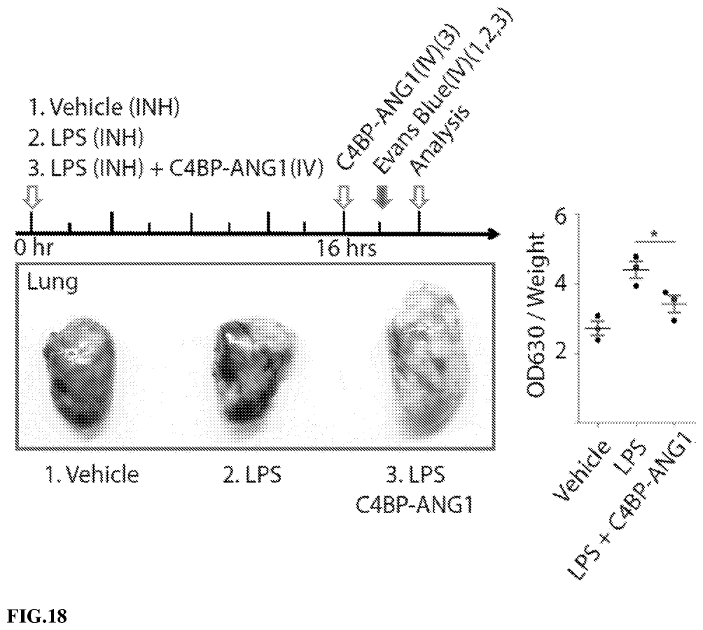

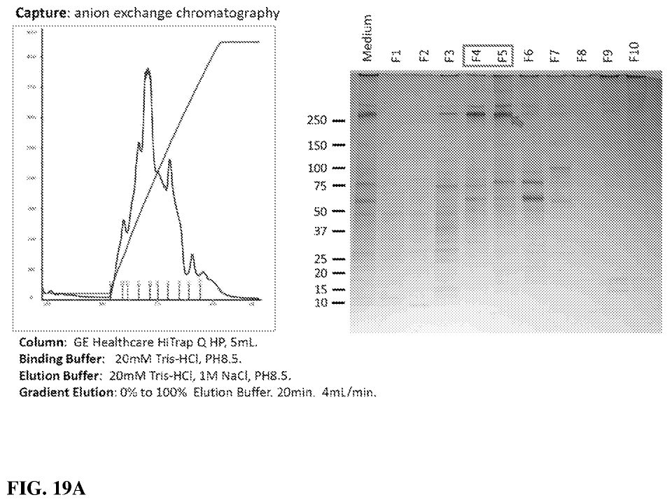

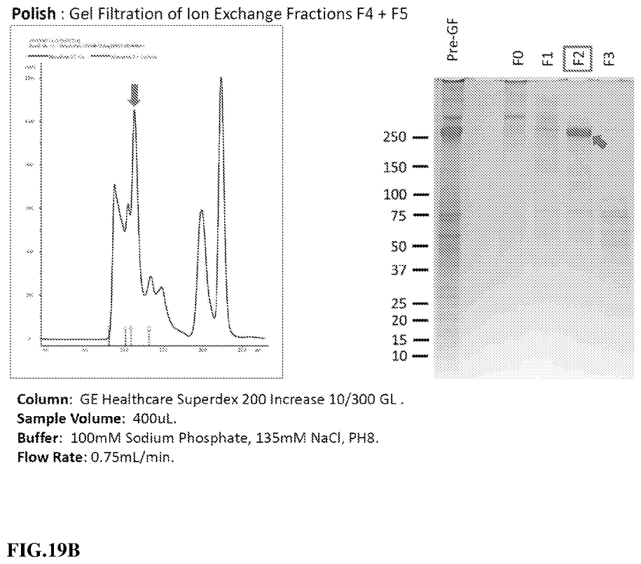

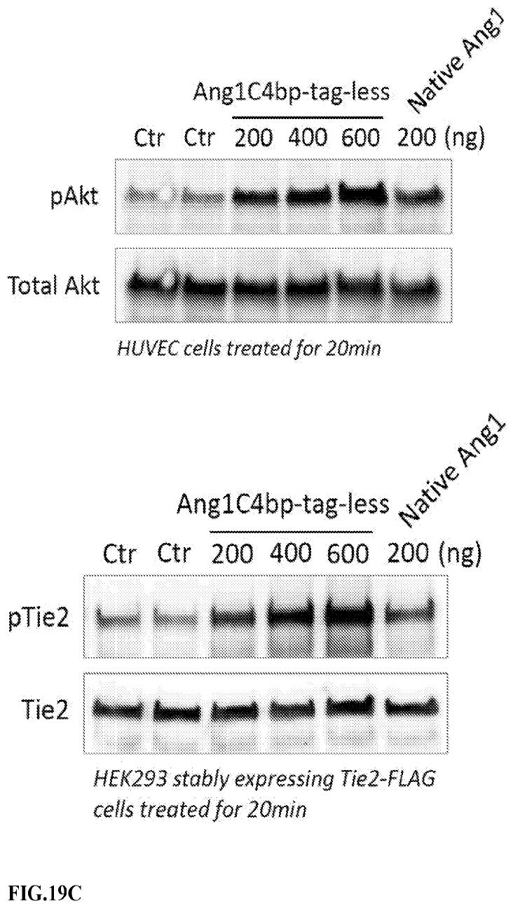

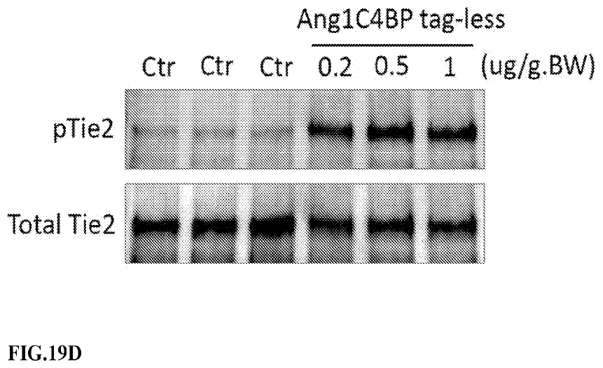

To determine the efficacy of C4BP-ANG1 in vivo, four different vascular permeability studies were conducted using Evans Blue dye (Miles assay) in BALB/c mice. Evens Blue dye has a very high affinity for serum albumin and its presence in interstitial space is indicative of blood vascular leak of protein. In the VEGF-induced subcutaneous permeability Miles assay, C4BP-ANG1 significantly reduced vascular leakage (FIG. 15). VEGF and C4BP-ANG1 were subcutaneously injected in mice either alone or together and Evans Blue dye was quantified by measuring optical density @630 nm (FIG. 15). Instead of local subcutaneous injection of C4BP-ANG1, intravenous injection of the biologic 30 minutes before subcutaneous VEGF also showed reduced vascular leakage with C4BP-ANG1 treatment (FIG. 16). Similarly, systemic intravenous injection of C4BP-ANG1 also reduced the severity of chemically induced vascular leakage (FIG. 17). In a pulmonary vascular permeability assay, intravenous injection of C4BP-ANG1 ameliorated vascular leakage in mice subjected to inhalation of bacterial lipopolysaccharide (LPS) to induce vascular leak in the lung (FIG. 18). Total Evans Blue dye extraction and measurement showed reduced leakage in mice treated with C4BP-ANG1 (FIG. 18). Collectively, these in vivo results demonstrate robust biological activity of C4BP-ANG1 and its vasculoprotective effect.

1. A chimeric polypeptide comprising the C-terminal domain of complement protein C4-binding protein (C4 bp) linked to the fibrinogen-like domain (FLD) of Angiopoietin (Ang).

2. The chimeric polypeptide of claim 1, wherein the C4 bp domain is at the N-terminus of the polypeptide and the Ang domain is at the C-terminus of the polypeptide thereby forming a C4 bp-Ang polypeptide.

3. The chimeric polypeptide of claim 1, wherein the Ang domain is at the N-terminus of the polypeptide and the C4 bp domain is at the C-terminus of the polypeptide thereby forming an Ang-C4 bp polypeptide.

4. The chimeric polypeptide of anyone of claims 1 through 3, wherein the Ang is Ang1 or Ang2.

5. The chimeric polypeptide of anyone of claims 1 through 4, wherein the C-terminal domain of C4 bp comprises SEQ ID NO.:1.

6. The chimeric polypeptide of anyone of claims 1 through 5, wherein the fibrinogen-like domain of Ang1 comprises SEQ ID NO.:2 and the fibrinogen-like domain of Ang2 comprises SEQ ID NO.:3

7. The chimeric polypeptide of anyone of claims 1 through 6, wherein the chimeric polypeptide comprises Ang1-C4 bp polypeptide; C4 bp-Ang1 polypeptide, C4 bp-Ang2, the HIS-tagged versions of the same, and/or the signal-peptide containing versions of the same.

8. The chimeric polypeptide of anyone of claims 1 through 7, wherein the polypeptide further comprises a signal peptide.

9. The chimeric polypeptide of claim 8, wherein the signal peptide is selected from the signal peptide of IL2 and the signal peptide of human CD33.

10. The chimeric polypeptide of anyone of claims 1 through 9, wherein the polypeptide comprises a signal peptide, and with and without a C-terminal label/tag.

11. The chimeric polypeptide of anyone of claims 1 through 9, wherein the polypeptide further comprises a linker peptide between the C4 bp domain and the Ang domain.

12. The chimeric polypeptide of anyone of claims 1 through 11, wherein the linker peptide is selected from a linker comprising the amino acid sequence GGGGS (SEQ ID NO: 4), EAAAK (SEQ ID NO: 32), PAPAP (SEQ ID NO: 33), AEAAAKEAAAKA (SEQ ID NO: 34), KESGSVSSEQLAQFRSLD (SEQ ID NO: 35), and EGKSSGSGSESKST (SEQ ID NO: 36).

13. The chimeric polypeptide of anyone of claims 1 through 12, wherein the polypeptide comprises a linker, without a C-terminal label.

14. The chimeric polypeptide of anyone of claims 1 through 13, wherein the polypeptide further comprises a N-terminal and/or C-terminal label.

15. The chimeric polypeptide of anyone of claims 1 through 14, wherein the label is selected from a poly-His, GST, MBP, Flag, CBP, and protein A label/tag.

16. The chimeric polypeptide of anyone of claims 1 through 15, wherein the polypeptide comprises the amino acid sequence of SEQ ID NO.: 9, 10, 11, 12, 13, 18, 20, 28, or 29.

17. The chimeric polypeptide of anyone of claims 1 through 16, further comprising an enterokinase cleavage site.

18. The chimeric polypeptide of anyone of claims 1 through 15, wherein the polypeptide comprises SEQ ID NO.: 15, 16, or 17.

19. The chimeric polypeptide of anyone of claims 1 through 18, wherein the polypeptide binds to Tie2, and/or activates Tie2, and/or activates phosphorylation of AKT in vitro and/or in vivo by at least one standard assay of the disclosure.

20. The chimeric polypeptide of anyone of claims 1 through 19, wherein the polypeptide reduces vascular permeability.

21. The chimeric polypeptide of claim 20, wherein the polypeptide reduces vascular permeability in the skin, eye, and/or lung.

22. The chimeric polypeptide of claim 20, wherein the polypeptide reduces increases in vascular permeability caused by VEGF, bacterial metabolites such as LPS, chemical toxin such as mustard oil, or other infections and chemical toxicities.

23. A nucleic acid encoding a polypeptide of anyone of claims 1 through 22.

24. The nucleic acid of claim 23, wherein the nucleic acid sequence has been codon-optimized for expression of the polypeptide in a bacterial, yeast, or mammalian cell.

25. The nucleic acid of anyone of claims 23 through 24, wherein the nucleic acid comprises SEQ ID NO.: 19 through 24.

26. A recombinant vector comprising a nucleic acid of anyone of claims 23 through 25.

27. The recombinant vector of claim 26, wherein the vector is an adenoviral vector, a retroviral vector, a lentiviral vector (etc.)

28. A protein complex comprising seven chimeric polypeptides, wherein the polypeptides are selected from the polypeptides of anyone of claims 1 through 22.

29. A cell comprising a polypeptide of anyone of claims 1 through 22, a nucleic acid of anyone of claims 23 through 25, a vector of anyone of claims 26 and 27, and/or a protein complex of claim 28.

30. A pharmaceutical composition comprising a nucleic acid of anyone of claims 23 through 25, a vector of anyone of claims 26 and 27, a protein complex of claim 28, and/or a cell of claim 29 and a pharmaceutically acceptable carrier, excipient, or diluent.

31. A method of reducing vascular permeability or leakage in a subject in need thereof comprising administering to the subject an effective amount of a polypeptide of anyone of claims 1 through 22, a cell of claim 29, a nucleic acid of anyone of claims 23 through 25, a vector of anyone of claims 26 and 27, a protein complex of claim 28, a cell of claim 29, and/or a pharmaceutical composition of claim 30.

32. The method of claim 30, wherein the vascular permeability or leakage has been increased in the skin, eye, lung, kidney, brain, liver, heart, and/or intestine

33. The method of claim 31, wherein the vascular permeability or leakage has been increased in response to increased levels of an agent selected from VEGF, infectious agents, toxic chemicals, etc.

34. A method of treating a disease or disorder accompanied by abnormal vascular permeability or leakage in a subject in need thereof comprising administering to the subject an effective amount of a polypeptide of anyone of claims 1 through 22, a cell of claim 29, a nucleic acid of anyone of claims 23 through 25, a vector of anyone of claims 26 and 27, and/or a protein complex of claim 28.

35. A method of treating a disease or disorder that responds to Tie2 activation or a disease or disorder that is associated with pathological angiogenesis in a subject in need thereof comprising administering to the subject an effective amount of a polypeptide of anyone of claims 1 through 22, a cell of claim 29, a nucleic acid of anyone of claims 23 through 25, a vector of anyone of claims 26 and 27, a protein complex of claim 28, and/or a pharmaceutical composition of claim 30, thereby activating Tie2.

36. The method of claim 34, wherein a disease or disorder that responds to Tie2 activation is any disease or disorder wherein at least one sign or the severity of a symptom, the frequency with which such a symptom is experienced by a patient, or both, is reduced or eliminated by Tie2 activation.

37. The method of anyone of claims 33 through 36, wherein the disorder is selected from cancer, ocular diseases or disorders, sepsis, inflammation, pneumonia, acute respiratory distress syndrome, renal dysfunction—acute kidney injury and chronic kidney disease, stroke, myocardial infarction, congestive heart failure, amyotropic lateral sclerosis, Alzheimer's disease, Huntington's disease, Parkinson's disease, peripheral neuropathies, traumatic brain injury, epilepsy, multiple sclerosis, and acute vascular destabilization and vascular leakage in diverse diseases such as viral hemorrhagic fevers (Ebola, Dengue, Hantavirus and Puumala), severe influenza, COVID-19, malaria, anthrax, chronic mycobacterial infection, and systemic capillary leak syndrome.

38. The method of claim 37, wherein the ocular disease or disorder is selected from the group consisting of age-related macular degeneration (AMD), macular degeneration, macular edema, diabetic macular edema (DME) (including focal, non-center DME and diffuse, center-involved DME), retinopathy, diabetic retinopathy (DR) (including proliferative DR (PDR), non-proliferative DR (NPDR), and high-altitude DR), other ischemia-related retinopathies, retinopathy of prematurity (ROP), retinal vein occlusion (RVO) (including central (CRVO) and branched (BRVO) forms), CNV (including myopic CNV), comeal neovascularization, a disease associated with comeal neovascularization, retinal neovascularization, a disease associated with retinal/choroidal neovascularization, pathologic myopia, von Hippel-Lindau disease, histoplasmosis of the eye, familial exudative vitreoretinopathy (FEVR), Coats' disease, Norrie Disease, Osteoporosis-Pseudoglioma Syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, conjunctivitis (including infectious conjunctivitis and non-infectious (e.g., allergic) conjunctivitis), Leber congenital amaurosis, uveitis (including infectious and non-infectious uveitis), choroiditis, ocular histoplasmosis, blepharitis, dry eye, traumatic eye injury, and Sjögren's disease.

39. The method of claim 37, wherein the ocular disease or disorder is glaucoma, AMD, or DME.

40. The method of any one of claims 31 through 39, wherein the polypeptide or pharmaceutical composition is administered intravitreally, ocularly, intraocularly, juxtasclerally, subtenonly, superchoroidally, topically, intravenously, intramuscularly, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, intraperitoneally, peritoneally, intraventricularly, subcutaneously, subconjunctivally, intravesicularly, mucosally, intrapericardially, intraumbilically, intraorbitally, orally, transdermally, by inhalation, by injection, by eye drop, by implantation, by infusion, by continuous infusion, by localized perfusion bathing target cells directly, by catheter, by lavage, in cremes, or in lipid compositions.

41. A method activating Tie2 in a subject in need thereof comprising administering to the subject an effective amount of a polypeptide of anyone of claims 1 through 22, a cell of claim 29, a nucleic acid of anyone of claims 23 through 25, a vector of anyone of claims 26 and 27, and/or a protein complex of claim 28.

42. The method of anyone of claims 31 through 41, wherein the method further comprises administering a second agent.

43. The method of claim 42, wherein the second agent is selected from an antibody, an anti-angiogenic agent, a cytokine, a cytokine antagonist, a corticosteroid, and an analgesic.

44. The method of claim 43, wherein the anti-angiogenic agent is a VEGF antagonist or a VE-PTP inhibitor.

45. The method of claim 44, wherein the VEGF antagonist is an anti-VEGF antibody, an anti-VEGF receptor antibody, a soluble VEGF receptor fusion protein, an aptamer, an anti-VEGF DARPin®, or a VEGFR tyrosine kinase inhibitor.

46. The method of claim 45, wherein the anti-VEGF antibody is ranibizumab (LUCENTIS®), RTH-258, or a bispecific anti-VEGF antibody.

47. The method of claim 46, wherein the bispecific anti-VEGF antibody is an anti-VEGF/anti-Ang2 antibody.

48. The method of claim 47, wherein the anti-VEGF/anti-Ang2 antibody is RG-7716.

49. The method of claim 45, wherein the soluble VEGF receptor fusion protein is aflibercept (EYLEA®).

50. The method of claim 45, wherein the aptamer is pegaptanib (MACUGEN®).

51. The method of claim 45, wherein the anti-VEGF DARPin® is abicipar pegol.

52. The method of claim 45, wherein the VEGFR tyrosine kinase inhibitor is selected from the group consisting of 4-(4-bromo-2-fluoroanilino)-6-methoxy-7-(1-methylpiperidin-4-ylmethoxy)quinazoline (ZD6474), 4-(4-fluoro-2-methylindol-5-yloxy)-6-methoxy-7-(3-pyrrolidin-1-ylpropoxy)quinazoline (AZD2171), vatalanib (PTK787), semaxaminib (SU5416), and SUTENT® (sunitinib).

53. The use of a polypeptide of anyone of claims 1 through 22, a cell of claim 29, a nucleic acid of anyone of claims 23 through 25, a vector of anyone of claims 26 and 27, and/or a protein complex of claim 28 in the manufacture of a medicament for the treatment of an ocular disease or disorder.

54. A polypeptide of anyone of claims 1 through 22, a cell of claim 29, a nucleic acid of anyone of claims 23 through 25, a vector of anyone of claims 26 and 27, and/or a protein complex of claim 28 for use in the treatment of an ocular disease or disorder.


SEQUENCE LISTING (36 sequences)

SEQ ID NO: 1 - protein, 57 aa, Homo sapiens
ETPEGCEQVLTGKRLMQCLPNPEDVKMALEVYKLSLEIEQLELQRDSARQSTLDKEL

SEQ ID NO: 2 - protein, 218 aa, Homo sapiens
KPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 3 - protein, 218 aa, Homo sapiens
ISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 4 - protein, 5 aa, Artificial Sequence
  source: /note=“Description of Artificial Sequence Synthetic peptide”
GGGGS

SEQ ID NO: 5 - protein, 20 aa, Artificial Sequence
  source: /note=“Description of Artificial Sequence Synthetic peptide”
MYRMQLLSCIALSLALVTNS

SEQ ID NO: 6 - protein, 16 aa, Artificial Sequence
  source: /note=“Description of Artificial Sequence Synthetic peptide”
MPLLLLLPLLWAGALA

SEQ ID NO: 7 - protein, 5 aa, Artificial Sequence
  source: /note=“Description of Artificial Sequence Synthetic peptide”
DDDDK

SEQ ID NO: 8 - protein, 286 aa, Artificial Sequence
  source: /note=“Description of Artificial Sequence Synthetic polypeptide”
KPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFGGGGSETPEGCEQVLTGKRLMQCLPNPEDVKMALEVYKLSLEIEQLELQRDSARQSTLDKELHHHHHH

SEQ ID NO: 9 - protein, 306 aa, Artificial Sequence
  source: /note=“Description of Artificial Sequence Synthetic polypeptide”
MYRMQLLSCIALSLALVTNSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFGGGGSETPEGCEQVLTGKRLMQCLPNPEDVKMALEVYKLSLEIEQLELQRDSARQSTLDKELHHHHHH

SEQ ID NO: 10 - protein, 286 aa, Artificial Sequence
  source: /note=“Description of Artificial Sequence Synthetic polypeptide”
ETPEGCEQVLTGKRLMQCLPNPEDVKMALEVYKLSLEIEQLELQRDSARQSTLDKELGGGGSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFHHHHHH

SEQ ID NO: 11 - protein, 306 aa, Artificial Sequence
  source: /note=“Description of Artificial Sequence Synthetic polypeptide”
MYRMQLLSCIALSLALVTNSETPEGCEQVLTGKRLMQCLPNPEDVKMALEVYKLSLEIEQLELQRDSARQSTLDKELGGGGSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFHHHHHH

SEQ ID NO: 12 - protein, 286 aa, Artificial Sequence
  source: /note=“Description of Artificial Sequence Synthetic polypeptide”
ETPEGCEQVLTGKRLMQCLPNPEDVKMALEVYKLSLEIEQLELQRDSARQSTLDKELGGGGSISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADFHHHHHH

SEQ ID NO: 13 - protein, 306 aa, Artificial Sequence
  source: /note=“Description of Artificial Sequence Synthetic polypeptide”
MYRMQLLSCIALSLALVTNSETPEGCEQVLTGKRLMQCLPNPEDVKMALEVYKLSLEIEQLELQRDSARQSTLDKELGGGGSISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADFHHHHHH

SEQ ID NO: 14 - protein, 292 aa, Artificial Sequence
  source: /note=“Description of Artificial Sequence Synthetic polypeptide”
HHHHHHGDDDDKKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFGGGGSETPEGCEQVLTGKRLMQCLPNPEDVKMALEVYKLSLEIEQLELQRDSARQSTLDKEL

SEQ ID NO: 15 - protein, 313 aa, Artificial Sequence
  source: /note=“Description of Artificial Sequence Synthetic polypeptide”
MYRMQLLSCIALSLALVTNSEHHHHHHGDDDDKKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFGGGGSETPEGCEQVLTGKRLMQCLPNPEDVKMALEVYKLSLEIEQLELQRDSARQSTLDKEL

SEQ ID NO: 16 - protein, 293 aa, Artificial Sequence
  source: /note=“Description of Artificial Sequence Synthetic polypeptide”
HHHHHHGGDDDDKETPEGCEQVLTGKRLMQCLPNPEDVKMALEVYKLSLEIEQLELQRDSARQSTLDKELGGGGSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 17 - protein, 314 aa, Artificial Sequence
  source: /note=“Description of Artificial Sequence Synthetic polypeptide”
MYRMQLLSCIALSLALVTNSEHHHHHHGGDDDDKETPEGCEQVLTGKRLMQCLPNPEDVKMALEVYKLSLEIEQLELQRDSARQSTLDKELGGGGSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 18 - protein, 274 aa, Artificial Sequence
  source: /note=“Description of Artificial Sequence Synthetic polypeptide”
MYRMQLLSCIALSLALVTNSEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFGGGGSETPEGCEQVLTGKRLMQCLPNPEDVKMALE

SEQ ID NO: 19 - protein, 302 aa, Artificial Sequence
  source: /note=“Description of Artificial Sequence Synthetic polypeptide”
MPLLLLLPLLWAGALAETPEGCEQVLTGKRLMQCLPNPEDVKMALEVYKLSLEIEQLELQRDSARQSTLDKELGGGGSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFHHHHHH

SEQ ID NO: 20 - protein, 254 aa, Artificial Sequence
  source: /note=“Description of Artificial Sequence Synthetic polypeptide”
EKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDFGGGGSETPEGCEQVLTGKRLMQCLPNPEDVKMALE

SEQ ID NO: 21 - DNA, 921 bp, Artificial Sequence
  source: /note=“Description of Artificial Sequence Synthetic polynucleotide”
atgtacaggatgcaactcctgtcttgcattgcactaagtcttgcacttgtcacgaattcgaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagattttggtggcggtggctcagagacccccgaaggctgtgaacaagtgctcacaggcaaaagactcatgcagtgtctcccaaacccagaggatgtgaaaatggccctggaggtatataagctgtctctggaaattgaacaactggaactacaaagggacagcgcaagacaatccactttggataaagaactacatcaccatcaccatcactaa

SEQ ID NO: 22 - DNA, 921 bp, Artificial Sequence
  source: /note=“Description of Artificial Sequence Synthetic polynucleotide”
atgtacagaatgcagctgctgtcctgtatcgccctgagcctggctctggtgaccaactctgagacaccagagggatgtgagcaggtgctgaccggcaagcgcctgatgcagtgcctgcccaatcctgaggatgtgaagatggccctggaggtgtacaagctgtccctggagatcgagcagctggagctgcagagggattccgcccggcagtctacactggacaaggagctgggaggaggaggcagcaagcctttcagggattgtgccgacgtgtatcaggctggctttaacaagtctggcatctacaccatctatatcaacaatatgccagagcccaagaaggtgttctgcaacatggacgtgaatggcggcggctggacagtgatccagcacagggaggatggcagcctggacttccagcggggctggaaggagtacaagatgggctttggcaacccatctggcgagtattggctgggcaatgagttcatctttgccatcacctcccagagacagtacatgctgcgcatcgagctgatggattgggagggcaatagggcttactctcagtatgaccggttccatatcggcaacgagaagcagaattaccggctgtatctgaagggacacaccggaacagctggcaagcagtccagcctgatcctgcatggcgccgatttttccaccaaggacgctgataacgacaattgcatgtgcaagtgcgccctgatgctgacaggaggatggtggttcgacgcttgcggaccaagcaacctgaatggcatgttttatacagctggccagaaccacggcaagctgaatggcatcaagtggcattacttcaagggcccttcttattccctgagatccaccacaatgatgatccgcccactggattttcaccatcaccatcaccattaa

SEQ ID NO: 23 - DNA, 909 bp, Artificial Sequence
  source: /note=“Description of Artificial Sequence Synthetic polynucleotide”
atgcctctgctgctgctgctgccactgctgtgggctggcgctctggccgagacaccagagggctgtgagcaggtgctgacaggcaagagactgatgcagtgcctgcccaaccctgaggatgtgaagatggctctggaggtgtacaagctgtctctggagatcgagcagctggagctgcagagggatagcgcccggcagtctaccctggacaaggagctgggaggaggaggctctaagcccttccgcgattgtgctgacgtgtatcaggccggctttaataagtccggcatctacaccatctatatcaacaatatgccagagcccaagaaggtgttctgcaacatggacgtgaatggcggcggctggacagtgatccagcacagggaggatggctccctggacttccagcggggctggaaggagtacaagatgggctttggcaacccttccggcgagtattggctgggcaatgagttcatctttgctatcacaagccagagacagtacatgctgcgcatcgagctgatggattgggagggcaacagggcctacagccagtatgaccggttccatatcggcaacgagaagcagaattacaggctgtatctgaagggccacaccggcacagctggcaagcagtccagcctgatcctgcatggcgctgacttctccaccaaggacgccgataacgacaattgcatgtgcaagtgcgctctgatgctgacaggaggatggtggttcgacgcttgtggaccatctaacctgaatggcatgttttataccgccggccagaaccacggcaagctgaatggcatcaagtggcattacttcaagggcccctcttattccctgagatccaccacaatgatgatccgccctctggattttcaccatcaccatcaccattaa

SEQ ID NO: 24 - DNA, 921 bp, Artificial Sequence
  source: /note=“Description of Artificial Sequence Synthetic polynucleotide”
atgtacagaatgcagctgctgagctgtatcgccctgtctctggctctggtgaccaactctgagacaccagagggctgtgagcaggtgctgaccggcaagcgcctgatgcagtgcctgcccaatcctgaggatgtgaagatggccctggaggtgtataagctgtccctggagatcgagcagctggagctgcagagagattctgctcgccagtccaccctggacaaggagctgggaggaggaggcagcatctctttcagagattgtgccgaggtgtttaagagcggccacaccacaaacggcatctacaccctgacattccctaattctacagaggagatcaaggcctattgcgacatggaggctggaggaggaggatggaccatcatccagaggcgggaggatggcagcgtggacttccagaggacatggaaggagtacaaagtgggctttggcaacccatctggcgagtattggctgggcaacgagttcgtgtcccagctgaccaatcagcagcggtacgtgctgaagatccatctgaaggattgggagggcaacgaggcctactctctgtatgagcacttttacctgtccagcgaggagctgaattatcgcatccatctgaagggcctgaccggcacagctggcaagatctcttccatctcccagcccggcaacgatttcagcaccaaggacggcgataatgacaagtgcatctgtaagtgctcccagatgctgacaggaggatggtggttcgacgcttgcggaccaagcaacctgaatggcatgtactatccccagaggcagaacacaaataagtttaatggcatcaagtggtactattggaagggctccggctatagcctgaaggccaccacaatgatgatccggcctgctgactttcaccatcaccatcaccattaa

SEQ ID NO: 25 - DNA, 942 bp, Artificial Sequence
  source: /note=“Description of Artificial Sequence Synthetic polynucleotide”
atgtacagaatgcagctgctgtcctgtatcgccctgagcctggctctggtgaccaactctgagcaccatcaccatcaccatggcgacgatgacgataagaagccattccgcgattgtgccgacgtgtatcaggctggctttaataagtccggcatctacaccatctatatcaacaatatgcccgagcctaagaaggtgttctgcaacatggatgtgaatggcggcggctggacagtgatccagcacagggaggatggcagcctggacttccagcggggctggaaggagtacaagatgggctttggcaacccctctggcgagtattggctgggcaatgagttcatctttgccatcacatcccagagacagtacatgctgcgcatcgagctgatggattgggagggcaacagggcttactctcagtatgaccggttccatatcggcaacgagaagcagaattacaggctgtatctgaagggacacaccggaacagctggcaagcagtccagcctgatcctgcatggcgccgatttttccaccaaggacgctgataacgacaattgcatgtgcaagtgcgccctgatgctgacaggaggatggtggttcgacgcttgcggaccaagcaacctgaatggcatgttttacaccgctggccagaaccacggcaagctgaatggcatcaagtggcattacttcaagggcccttcttattccctgagaagcaccacaatgatgatcaggcctctggattttggaggaggaggctctgagacaccagagggatgtgagcaggtgctgacaggcaagcggctgatgcagtgcctgccaaatcccgaggacgtgaagatggccctggaggtgtataagctgtccctggagatcgagcagctggagctgcagagggattccgcccggcagtctacactggacaaggagctgtaa

SEQ ID NO: 26 - DNA, 945 bp, Artificial Sequence
  source: /note=“Description of Artificial Sequence Synthetic polynucleotide”
atgtacagaatgcagctgctgtcctgtatcgccctgagcctggctctggtgaccaactctgagcaccatcaccatcaccatggcggcgacgatgacgataaggagacacccgagggctgtgagcaggtgctgacaggcaagcgcctgatgcagtgcctgcccaatcctgaggatgtgaagatggccctggaggtgtacaagctgtccctggagatcgagcagctggagctgcagagggattccgcccggcagtctacactggacaaggagctgggaggaggaggcagcaagcctttcagggattgtgccgacgtgtatcaggctggctttaacaagtctggcatctacaccatctatatcaacaatatgccagagcccaagaaggtgttctgcaacatggacgtgaatggcggcggctggacagtgatccagcacagggaggatggcagcctggacttccagcggggctggaaggagtacaagatgggctttggcaacccatctggcgagtattggctgggcaatgagttcatctttgccatcacctcccagagacagtacatgctgcgcatcgagctgatggattgggagggcaatagggcttactctcagtatgaccggttccatatcggcaacgagaagcagaattaccggctgtatctgaagggacacaccggaacagctggcaagcagtccagcctgatcctgcatggcgccgatttttccaccaaggacgctgataacgacaattgcatgtgcaagtgcgccctgatgctgacaggaggatggtggttcgacgcttgcggaccaagcaacctgaatggcatgttttatacagctggccagaaccacggcaagctgaatggcatcaagtggcattacttcaagggcccttcttattccctgagatccaccacaatgatgatccgcccactggatttttaa

SEQ ID NO: 27 - DNA, 906 bp, Artificial Sequence
  source: /note=“Description of Artificial Sequence Synthetic polynucleotide”
atgtacagaatgcagctgctgtcctgtatcgccctgagcctggctctggtgaccaactctgagaagccattccgcgattgtgccgacgtgtatcaggctggctttaataagtccggcatctacaccatctatatcaacaatatgcccgagcctaagaaggtgttctgcaacatggatgtgaatggcggcggctggacagtgatccagcacagggaggatggcagcctggacttccagcggggctggaaggagtacaagatgggctttggcaacccctctggcgagtattggctgggcaatgagttcatctttgccatcacatcccagagacagtacatgctgcgcatcgagctgatggattgggagggcaacagggcttactctcagtatgaccggttccatatcggcaacgagaagcagaattacaggctgtatctgaagggacacaccggaacagctggcaagcagtccagcctgatcctgcatggcgccgatttttccaccaaggacgctgataacgacaattgcatgtgcaagtgcgccctgatgctgacaggaggatggtggttcgacgcttgcggaccaagcaacctgaatggcatgttttacaccgctggccagaaccacggcaagctgaatggcatcaagtggcattacttcaagggcccttcttattccctgagaagcaccacaatgatgatcaggcctctggattttggaggaggaggctctgagacaccagagggatgtgagcaggtgctgacaggcaagcggctgatgcagtgcctgccaaatcccgaggacgtgaagatggccctggaggtgtataagctgtccctggagatcgagcagctggagctgcagagggattccgcccggcagtctacactggacaaggagctgtaa

SEQ ID NO: 28 - protein, 280 aa, Artificial Sequence
  source: /note=“Description of Artificial Sequence Synthetic polypeptide”
ETPEGCEQVLTGKRLMQCLPNPEDVKMALEVYKLSLEIEQLELQRDSARQSTLDKELGGGGSKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 29 - protein, 280 aa, Artificial Sequence
  source: /note=“Description of Artificial Sequence Synthetic polypeptide”
ETPEGCEQVLTGKRLMQCLPNPEDVKMALEVYKLSLEIEQLELQRDSARQSTLDKELGGGGSISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 30 - protein, 6 aa, Artificial Sequence
  source: /note=“Description of Artificial Sequence Synthetic 6xHis tag”
HHHHHH

SEQ ID NO: 31 - protein, 4 aa, Artificial Sequence
  source: /note=“Description of Artificial Sequence Synthetic peptide”
GGGS

SEQ ID NO: 32 - protein, 5 aa, Artificial Sequence
  source: /note=“Description of Artificial Sequence Synthetic peptide”
EAAAK

SEQ ID NO: 33 - protein, 5 aa, Artificial Sequence
  source: /note=“Description of Artificial Sequence Synthetic peptide”
PAPAP

SEQ ID NO: 34 - protein, 12 aa, Artificial Sequence
  source: /note=“Description of Artificial Sequence Synthetic peptide”
AEAAAKEAAAKA

SEQ ID NO: 35 - protein, 18 aa, Artificial Sequence
  source: /note=“Description of Artificial Sequence Synthetic peptide”
KESGSVSSEQLAQFRSLD

SEQ ID NO: 36 - protein, 14 aa, Artificial Sequence
  source: /note=“Description of Artificial Sequence Synthetic peptide”
EGKSSGSGSESKST